(19) United States
(12) Patent Application Publication
(10) Pub. No.: US 2026/0284647 A1
(43) Pub. Date: Sep. 24, 2026

(54) Organic Photoredox Catalysts, Compositions, and Conversion Processes

(71) Applicants: Colorado State University Research Foundation, Fort Collins, CO (US); The Regents of the University of Colorado, a body corporate, Denver, CO (US)

(72) Inventors: Garret MIYAKE, Fort Collins, CO (US); Amreen BAINS, Fort Collins, CO (US); Niels DAMRAUER, Fort Collins, CO (US); Arindam SAU, Fort Collins, CO (US)

(21) Appl. No.: 19/573,904

(22) Filed: Mar. 20, 2026

Related U.S. Application Data
(60) Provisional application No. 63/775,664, filed on Mar. 21, 2025.
(60) Provisional application No. 63/972,799, filed on Jan. 30, 2026.
(60) Provisional application No. 63/976,929, filed on Feb. 5, 2026.

(51) Int. Cl. B01J 31/04; B01J 35/39; B01J 37/04; C07B 31/00; C07C 5/08; C07C 17/354; C07C 29/17; C07C 37/00; C07C 41/20; C07C 45/62; C07C 51/36; C07C 67/303; C07C 209/70; C07C 209/72; C07C 213/08; C07C 241/02; C07C 253/30; C07C 269/06; C07D 209/08; C07D 215/06; C07D 219/02; C07D 307/79; C07D 307/87; C07D 311/02; C07D 333/70; C07D 453/04; C07J 1/00; C07J 33/00

(52) U.S. Cl. CPC B01J 31/04; B01J 35/39; B01J 37/04; C07B 31/00; C07C 5/08; C07C 17/354; C07C 29/17; C07C 37/003; C07C 41/20; C07C 45/62; C07C 51/36; C07C 67/303; C07C 209/70; C07C 209/72; C07C 213/08; C07C 241/02; C07C 253/30; C07C 269/06; C07D 209/08; C07D 215/06; C07D 219/02; C07D 307/79; C07D 307/87; C07D 311/02; C07D 333/70; C07D 453/04; C07J 1/0051; C07J 33/002; B01J 2231/64; C07C 2601/16; C07C 2602/08; C07C 2602/10; C07C 2602/24; C07C 2602/28; C07C 2602/42; C07C 2603/18; C07C 2603/20; C07C 2603/24; C07C 2603/26

(57) ABSTRACT

Aspects described herein generally relate to a new class of photoredox catalysts and catalyst compositions that include the photoredox catalysts. In an aspect, a catalyst composition is provided. The catalyst composition comprises a metal free organic photoredox catalyst comprising a coronenyl compound. Aspects described herein also generally relate to conversion processes using the photoredox catalysts and catalyst compositions. In an aspect, a conversion process is provided. The conversion process includes forming a reaction mixture comprising a substrate and a catalyst composition comprising a reductant and an organic photoredox catalyst described herein. The conversion process further includes exposing the reaction mixture to light to convert the substrate, under conversion conditions, to a conversion product.

### CROSS-REFERENCE TO RELATED APPLICATIONS

This application claims benefit of and priority to U.S. Provisional Patent Application Ser. No. 63/775,664, filed on Mar. 21, 2025, U.S. Provisional Patent Application Ser. No. 63/972,799, filed on Jan. 30, 2026, and U.S. Provisional Patent Application Ser. No. 63/976,929, filed on Feb. 5, 2026, each of which is incorporated herein by reference in its entirety.

### GOVERNMENT SUPPORT

This invention was made with government support under grant 2318141 awarded by the National Science Foundation Center for Chemical Innovation Center for Sustainable Photoredox Catalysis (SuPRCat). The government has certain rights in the invention.

### FIELD

Aspects described herein generally relate to a new class of photoredox catalysts and catalyst compositions. Aspects described herein also generally relate to conversion processes using the photoredox catalysts and catalyst compositions.

### BACKGROUND

Traditional approaches for reducing aromatic rings to sp³-hybridized molecules include the Birch reduction, which typically utilizes pyrophoric lithium or sodium metal as reductants in condensed ammonia. Unlike catalytic hydrogenation, the Birch reduction does not fully reduce the arene, and instead reduces arenes to 1,4-cyclohexadienes. Although effective, these harsh and potentially unsafe reaction conditions deter implementation, especially at larger scales, and have poor functional group tolerance. More recently, photoredox catalysis driven by visible light has been investigated for its use in a variety of chemical reactions due to its ability to enable transformations under milder reaction conditions. However, conventional visible-light photoredox catalysis is constrained by at least the thermodynamic limits of photon energy and inefficiencies arising from unproductive back electron transfer, both of which become particularly pronounced in thermodynamically demanding reactions, such as the reduction of arenes and heteroarenes. In addition, conventional photoredox catalysts such as anionic photoredox catalysts, dianionic photoredox catalysts, and closed-shell anionic photoredox catalysts are challenged by narrow energy gaps, inefficiencies at reducing unactivated arenes, or are difficult to form without tailored photoredox catalyst structures.

There is a need for new catalysts and catalyst compositions. There is also a need for conversion processes using the catalysts and catalyst compositions.

### SUMMARY

Aspects described herein generally relate to a new class of photoredox catalysts and catalyst compositions that include the photoredox catalysts. Aspects described herein also generally relate to conversion processes using the photoredox catalysts and catalyst compositions. Photoredox catalysts, catalyst compositions, methodologies, and processes described herein may be utilized to transform a large scope of substrates to conversion products. For example, and unlike conventional photoredox catalysis, aspects of the present disclosure may be used to reduce aromatic rings and heteroaromatic rings and may be used to selectively reduce non-aryl and non-heteroaryl functional groups in the presence of aryl and heteroaryl groups on the same molecule. Aspects described herein may also be utilized for reductive detosylation, debenzylation, dephosphonation, degradation of a quaternary ammonium salts, hydrodefluorination, cross-coupling, cyclization, cleavage of sulfones, reductive alkylation, ester fragmentation, and/or lignin fragmentation, among other useful chemical reactions. Unlike conventional technologies for reductions of arenes, aspects described herein may have broad applicability to transform substrates under visible light and may be used to reduce unactivated arenes (for example, benzene). Further aspects described herein may facilitate chemical reactions at temperatures significantly below those of conventional technologies and without the use of metals or pyrophoric species.

In an aspect, a catalyst composition is provided. The catalyst composition includes a metal free organic photoredox catalyst comprising a coronenyl compound represented by formula (I):

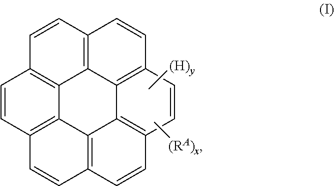

wherein, in formula (I): each H is a hydrogen atom; each R^{A} is, independently, a C1-C40 unsubstituted hydrocarbyl, a C1-C40 substituted hydrocarbyl, a functional group comprising at least one element from group 13-17 of the periodic of the elements, or two or more R^{A} join together to join together to form a monocyclic ring structure or a polycyclic ring structure; x of formula (I) is from 1 to 11, and when x is more than 1, each R^{A} is the same or different; y of formula (I) is from 1 to 11; and x+y is 12.

In another aspect, a conversion process is provided. The conversion process includes forming a reaction mixture comprising a substrate and a catalyst composition comprising a reductant and an organic photoredox catalyst described herein. The conversion process further includes exposing the reaction mixture to light to convert the substrate, under conversion conditions, to a conversion product.

In another aspect, a conversion process is provided. The conversion process includes forming a reaction mixture comprising a fluorine-containing substrate and a catalyst composition comprising a reductant and an organic photoredox catalyst described herein. The conversion process further includes exposing the reaction mixture to light to convert the substrate, under conversion conditions, to a conversion product.

### DETAILED DESCRIPTION

Aspects described herein generally relate to a new class of photoredox catalysts, catalyst compositions that include the photoredox catalysts, and to conversion processes using the catalysts and catalyst compositions. As described herein, the inventors discovered photoredox catalysts, catalyst compositions, and conversion processes that may be utilized to reduce aryls and heteroaryls, including unactivated aryls such as benzene, using mild and safe conditions. The catalysts, catalyst compositions, and conversion processes are applicable to a wide range of substrates and may be used for a wide variety of chemical reactions.

Generally, photoexcitation of a photoredox catalyst (PC) may form an excited state (PC*) that is both more reducing and oxidizing than the ground state and may mediate electron-transfer derived catalysis in either manifold if thermodynamically compatible with the substrate and the PC* lifetime is sufficiently long-lived to engage in bimolecular collisions. The use of visible light is desirable to selectively excite the PC and avoid undesirable side-reactivity that occurs with higher energy light. However, a visible-wavelength photon places thermodynamic constraints on the reactivity, limiting the scope of chemical transformations. For example, using photoredox catalysis for reduction of inert planar arenes to three-dimensional sp³-hybridized molecules is a strongly desired transformation but has only been achieved with systems that achieve low catalytic activity and are restricted to activated arenes, both of which are undesirable.

Conventional catalysts capable of reducing inert substrates include anionic PCs. The most common anionic PCs studied are radical anions (PC*⁻) which are typically generated using sacrificial electron donors or electrochemical methods. However, these species are disadvantaged by narrow energy gaps in the doublet manifold (Do-Di) that give rise to short excited state lifetimes incommensurate with electron transfer initiated by bimolecular collisions. Dianionic PCs (PC²⁻) may have the ability for increased excited state reduction potential and lifetime. However, their formation has been difficult to achieve without tailored PC structures. Closed-shell anionic PCs (PC⁻) generated from deprotonation of neutral PCs is another conventional approach but are inefficient at reducing unactivated arenes such as benzene. Recently, 2e⁻/1H⁺ reduced PC species ([PC⁻H]⁻) have been presented as potential candidates for challenging photocatalytic systems. Such closed-shell, effectively hydride-reduced PCs can be generated through two sequential one-electron reductions followed by protonation or hydrogen atom transfer after the first reduction. These species bear the benefits of an anionic PC but possess a larger S₀-S₁ gap, resulting in multi-nanosecond range excited state lifetimes. However, despite these promising attributes, efficient and robust catalyst systems for arene reductions based on [PC⁻H]⁻ have not been discovered.

Even if a potent reducing PC system is established, an arguably even more challenging hurdle to successful catalysis is unproductive back electron transfer (BET). BET limits photocatalytic productivity by shunting desired electron transfers and has generally been considered an inherent shortcoming in photoredox catalysis. For example, when a neutral PC* reduces a substrate (S), charge separation produces an oppositely charged PC radical cation (PC*⁺) and substrate radical anion (S*⁻) with Coulombic attraction. In the absence of cage escape, BET can reverse the reaction and erode product formation. Although absent of Coulombic attractions, a BET scenario persists in charge-shift reactions involving an anionic PC⁻* that transfers an electron to S, producing a neutral radical (PC*) and an S*⁻. BET is particularly prevalent with arenes that are difficult to reduce, as the stored potential provides a driving force for BET to the oxidized catalyst (PC*‘ or PC’). To bypass these challenges, many photoredox catalyzed reactions incorporate irreversible chemical events such as those that occur with sacrificial donors following oxidation or when substrates fragment, such as organohalides, which, upon reduction undergo carbon-halogen bond breaking to effectively compete with BET, even in the absence of cage escape.

Arene reduction has been attempted with super-reducing photoredox catalysts, such as benzo[ghi]perylene imide compounds, in conjunction with an electron source. Despite possessing an excited state reduction potential sufficient to reduce benzene, this catalyst system was only able to achieve very low yields, if any, of arene reduction products even when using require multiple catalyst loadings and long reaction times. Overall, super-reducing photoredox catalysts are either inefficient or do not work for various substrates.

In contrast, photoredox catalysts and catalyst compositions described herein may be utilized, for example, to reduce aryls and heteroaryls, including unactivated aryls such as benzene, using mild and safe conditions. In some examples, the photoredox catalyst include a benzo[a]coronene moiety such as a benzo[a]coronene diester, which demonstrated significantly improved catalytic activity for arene reduction relative to conventional photoredox approaches. The enhanced activity may be attributed to the highly reducing excited state of this catalyst (−3.9 V vs Fc⁺/Fc), suppression of BET through a deprotonation mechanism of the catalyst itself, or both. Aspects described herein may be utilized to overcome obstacles to conventional approaches and may to drive chemical transformations that require super-reducing capabilities. This advancement may be accomplished by coupling the energy of two photons into a single high-energy chemical reduction, effectively surpassing the inherent thermodynamic constraints of a single photon while inefficiencies from BET may be mitigated through a unique proton-coupled electron transfer mechanism embedded in the catalyst design. The super-reducing capabilities of photoredox catalysts and catalyst compositions described herein are demonstrated through efficient application in a broad scope of challenging reductions and other chemical transformations.

The use of headings is for purposes of convenience only and does not limit the scope of the present disclosure. Aspects described herein can be combined with other aspects. For example, features and elements of one described aspect may be combined with other aspects within the scope of the present disclosure.

### Catalysts and Catalyst Compositions

Aspects described herein relate to a new class of catalysts and catalyst compositions. As used herein, a “composition” may include component(s) of the composition, reaction product(s) of two or more components of the composition, a remainder balance of remaining starting component(s), or combinations thereof. Catalyst compositions of the present disclosure include an organic photoredox catalyst, also referred to herein as photoredox catalysts. In contrast to conventional technologies, the organic photoredox catalyst may be metal free. The organic photoredox catalyst may include a coronenyl compound represented by formula (I):

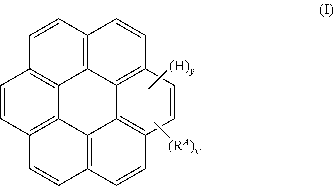

In formula (I), H is a hydrogen atom. Each R^{A} of formula (I) may be, independently, a C1-C40 unsubstituted hydrocarbyl, a C1-C40 substituted hydrocarbyl, a functional group comprising at least one element from Group 13-17 of the periodic table of the elements, or two or more R^{A} join together to join together to form a monocyclic ring structure or a polycyclic ring structure. In formula (I), x may be from 1 to 11, such as from 1 to 10, such as from 1 to 8; y is from 1 to 11; and x+y may be equal to 12. When x of formula (I) is more than 1, each R^{A} may be the same or different. C1-C40 unsubstituted hydrocarbyls for R^{A} of formula (I) may include C1-C20, C1-C16, C1-C12, C1-C8, or C1-C4 unsubstituted hydrocarbyls, for example, alkyls such as methyl, ethyl, propyl, or butyl, alkenyls such as such as ethenylene (—CH═CH—), propenylene (—CH═CHCH₂—), 2-butenylene (—CH₂CH═CHCH₂—), alkynyls such as propargyl, or aryls such as phenyl (Ph) or naphthyl. C1-C40 substituted hydrocarbyls may include C1-C20, C1-C16, C1-C12, C1-C8, or C1-C4 substituted hydrocarbyls where at least one hydrogen of the hydrocarbyl is substituted with at least one heteroatom or heteroatom-containing group, such as a halogen (F, Cl, Br, or I) or one or more elements from Group 13-16 of the periodic table of the elements such as F, Cl, Br, I, O, N, P, S, B, Si, and the like, such as C(O)R*, C(C)NR*₂, C(O)OR*, NR*₂, OR*, PR*₂, SR*, SOx (where x=2 or 3), BR*₂, SiR*₃, and the like, where R* is, independently, hydrogen or hydrocarbyl (such as C1-C4 hydrocarbyl), or where at least one heteroatom has been inserted within the R group or the unsubstituted hydrocarbyl.

The functional group comprising at least one element from Group 13-17 of the periodic table of the elements may include a halogen (F, Cl, Br, or I), O, N, Se, Te, P, As, Sb, S, B, Si, Ge, Sn, Pb, and the like, such as C(O)R*, C(O)NR*₂, C(O)OR*, NR*₂, OR*, SeR*, TeR*, PR*₂, AsR*₂, SbR*₂, SR*, SOx (where x=2 or 3), BR*₂, SiR*₃, GeR*₃, SnR*₃, PbR*₃, and the like, wherein each R* is, independently, hydrogen or hydrocarbyl (such as a C1-C4 hydrocarbyl). Non-limiting examples of functional groups that replace one or more hydrogen atoms may, independently, include —OH, —F, —Cl, —Br, —I, —NH₂, —NO₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

Illustrative, but non-limiting, C1-C40, C1-C20, C1-C16, C1-C12, C1-C8, or C1-C4 substituted hydrocarbyls for R^{A} of formula (I) may include esters (e.g., CO₂Me, CO₂Et, and the like), ketones, amides, amines, nitriles, ethers, alcohols, thioesters, thioketones, sulfides, thiols, or halides, such as esters, ketones, amides, amines, or nitriles. Ethers (also referred to as “alkoxyls” or “alkoxy”) refer to a hydrocarbyl ether or aryl ether group. Examples of suitable alkoxyls for R^{A} of formula (I) may include C1-C20 alkoxyls or C1-C10 alkoxyls such as, but not limited to, methoxy (OMe), ethoxy (OEt), n-propoxy, isopropoxy, n-butoxy, isobutoxy, sec-butoxy, tert-butoxy, phenoxy, etc., such as OMe, or OEt.

The organic photoredox catalyst may include a benzo[a]coronenyl compound represented by formula (I-A):

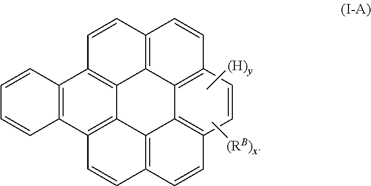

H (hydrogen) and R^{B} of formula (I-A) may be the same as that described herein for H and R^{A} of formula (I). In formula (I-A), x may be from 1 to 13, such as from 1 to 12, such as from 1 to 10; y may be from 1 to 13, such as from 1 to 8, such as from 1 to 6; and x+y is equal to 14. When x of formula (I-A) is more than 1, each R^{B} may be the same or different.

The organic photoredox catalyst of formula (I-A) may include a benzo[a]coronenyl compound represented by formula (I-A-1):

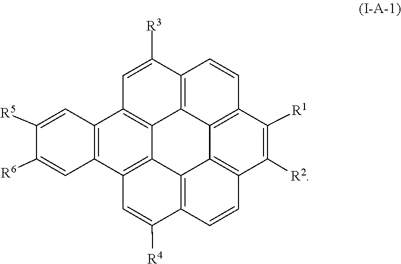

Each of R¹ and R² of formula (I-A-1) may be, independently, a C1-C40 substituted hydrocarbyl, halogen, or R¹ and R² join together to form a monocyclic or polycyclic ring structure (e.g., a C4-C40 monocyclic ring structure or a C4-C40 polycyclic ring structure).

C1-C40 substituted hydrocarbyls for R¹ and R² of formula (I-A-1) may include those C1-C40 substituted hydrocarbyls described herein for R^{A} of formula (I), such as C1-C20, C1-C16, C1-C12, C1-C8, or C1-C4 substituted hydrocarbyls, such as esters (e.g., CO₂Me, CO₂Et, and the like), ketones, amides, amines, nitriles, ethers (alkoxyls), alcohols, thioesters, thioketones, sulfides, thiols, or halides, such as esters, ketones, amides, amines, nitriles, or ethers.

C4-C40 monocyclic and polycyclic ring structures may include substituted C4-C40 monocyclic and polycyclic ring structures, such as ring structures having substituted hydrocarbyl substituents. Substituted C4-C40 monocyclic and polycyclic ring structures may include C4-C30 monocyclic and polycyclic ring structures, such as C4-C20 monocyclic and polycyclic ring structures, such as a cyclic diester or a diimide. C4-C40 monocyclic and polycyclic ring structures may be fully saturated, partially unsaturated, or fully unsaturated, aromatic or not aromatic.

Each of R³ and R⁴ of formula (I-A-1) may be, independently, hydrogen or a C1-C40 unsubstituted hydrocarbyl. C1-C40 unsubstituted hydrocarbyls for R³ and R⁴ of formula (I-A-1) may include those C1-C40 unsubstituted hydrocarbyls described herein for R^{A} of formula (I), such as C1-C40, C1-C20, C1-C16, C1-C12, C1-C8, or C1-C4 unsubstituted hydrocarbyls, e.g., alkyls such as methyl, ethyl, propyl, or butyl, or aryls such as phenyl (Ph) or naphthyl.

Each of R⁵ and R⁶ of formula (I-A-1) may be, independently, hydrogen, C1-C40 unsubstituted hydrocarbyl, or C1-C40 substituted hydrocarbyl. C1-C40 unsubstituted hydrocarbyls and C1-C40 substituted hydrocarbyls for R⁵ and R⁶ of formula (I-A-1) may include those described herein for R^{A} of formula (I). Illustrative, but non-limiting examples of C1-C20 substituted hydrocarbyls for R⁵ and R⁶ of formula (I-A-1) may include a C1-C20 alkoxyl, such as a C1-C10 alkoxyl. Examples of suitable C1-C20 or C1-C10 alkoxyls may include those alkoxyls described herein with respect to R^{A} of formula (I).

Illustrative, but non-limiting, examples of organic photoredox catalysts of formula (I-A-1) described herein may include one or more benzo[a]coronenyl compounds Cat-1, Cat-2, Cat-3, or Cat-4:

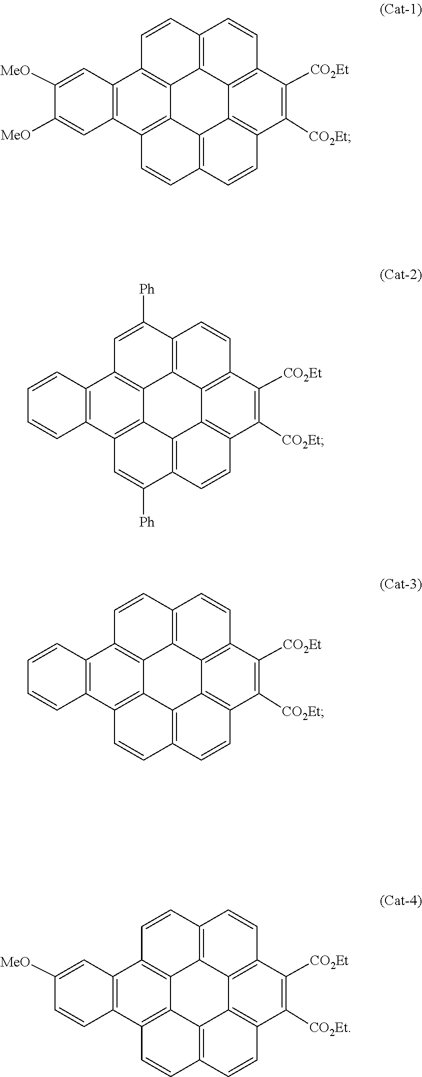 [SMILES: CCOC(=O)c1c(C(=O)OCC)c2ccc3ccc4c5cc(OC)c(OC)cc5c5ccc6ccc1c1c6c5c4c3c21 ; CCOC(=O)c1c(C(=O)OCC)c2ccc3c(-c4ccccc4)cc4c5ccccc5c5cc(-c6ccccc6)c6ccc1c1c2c3c4c5c61 ; CCOC(=O)c1c(C(=O)OCC)c2ccc3ccc4c5ccccc5c5ccc6ccc1c1c6c5c4c3c21 ; CCOC(=O)c1c(C(=O)OCC)c2ccc3ccc4c5cc(OC)ccc5c5ccc6ccc1c1c6c5c4c3c21]

The organic photoredox catalyst may be characterized as a catalyst that accepts electrons, donates electrons, accepts protons, donates protons, or combinations thereof, for example, a two-electron, 1-proton transfer catalyst. The organic photoredox catalyst may be characterized as a “super-reducing catalyst,” facilitating reductions of stable, difficult-to-reduce or difficult-to-break bonds at, for example, room temperature.

While not wishing to be bound by any theory, the inventors believe that organic photoredox catalysts of the present disclosure may perform in the follow manner. The organic photoredox catalysts achieve a 2 electron/1 proton state by sequentially coupling multiple photon absorptions to achieve a super-reducing capability. This advancement allows for a single photon to photoexcite the catalyst, and produce high energy chemical reductions due to its highly energized state. From this highly energized state, the organic photoredox catalyst may donate electron(s) to a substrate (a material that is to be converted in a reaction, also referred to as a starting material). An innovation with organic photoredox catalysts described herein may include a proton coupled transfer, for example, the organic photoredox catalyst may coordinate a proton transfer event with the electron transfer. The proton transfer event removes one of the protons, or accompanies an electron transfer reaction elsewhere in the reduction, thereby helping to mitigate or prevent a fast “back electron transfer” where the electrons would return to the starting molecule and reduce the efficiency of a catalyst system

Catalyst compositions described herein may further include a reductant. The reductant may serve as an electron donor. Suitable reductants may include amines (such as trialkyl or triaryl amines), quaternary ammoniums, hydroxides, carboxylic acids, or salts thereof, or combinations thereof, such as tetramethylammonium hydroxide (TMAOH, Me₄NOH), trimethylamine (NMe₃), N,N-diisopropylethylamine (DIPEA), tetra-n-butylammonium fluoride (TBAF, nBu₄NF), tetra-n-butylammonium chloride (TBACl, nBu₄NCl), tetra-n-butylammonium bromide (TBABr, nBu₄NBr), a formate salt (a salt of formic acid, such as sodium formate). Additionally, or alternatively, the reductant may include the use of an electrode. For example, the reductant may be delivered using electrochemistry. Here, the electrode serves as a source of electrons. When utilized, an anode and a cathode may be immersed in the reaction mixture and an electric field may be applied to the reaction mixture by use of the anode and the cathode to promote or cause a reaction to occur. Other set-ups for the electrode are contemplated. The electric field applied may have a voltage that is in a range from about −2 V to about −4, such as from about −2 V to about −3 vs ferrocene/ferrocenium (Fc/Fc⁺).

The catalyst composition may include a solvent. The solvent may include tetrahydrofuran (THF), tert-amyl alcohol (also referred to as tert-AmylOH, t-AmylOH, or 2-methylbutan-2-ol), dimethylformamide (DMF), dichloromethane (DCM), toluene, dimethylacetamide (DMAc), acetonitrile (CH₃CN), dimethyl sulfoxide (DMSO), water (H₂O), or combinations thereof. The solvent may include a solvent system, which is a mixture of two or more solvents in suitable proportions. For example, a mixture of t-AmylOH and THF, such as a mixture, by volume, in a range from about 0.1:1 to about 1:0.1, such as about 1:1 (t-AmylOH:THF) may be utilized. As another example, a mixture of H₂O and THF, such as a mixture, by volume, in a range from about 0.05:1.0 to about 1.0:0.05, such as about 0.1:0.9 (H₂O:THF), may be utilized.

The catalyst composition may be utilized to catalyze the reduction of bonds in a substrate, catalyze the breaking of bonds in a substrate, catalyze reactions involving a substrate, etc., or combinations thereof. Such catalysis may be performed under conversion conditions described herein. A “substrate” refers to a starting material or reactant, for example, it is a substance that is acted upon and transformed into a conversion product. A “conversion product” refers to the product of the reaction where the substrate is transformed, for example, a new chemical substance(s) that forms when the substrate undergoes a reaction. The conversion product may include byproducts, coproducts, or combinations thereof.

For example, the catalyst composition may be utilized to reduce a substrate under conversion conditions. Illustrative, but non-limiting substrates for reduction may include an aryl or a heteroaryl. The aryl or heteroaryl may be unsubstituted, only including hydrogen substituents. Alternatively, the aryl or heteroaryl may be substituted, for example, with a non-hydrogen substituent, such as an alkenyl group (e.g., III-41a, III-43a), an alkynyl group (e.g., III-109a), a substituted hydrocarbyl (e.g., III-42), an imine group (—N═C(H)—) (e.g., III-44a, -45a, -46a, and -47a), an azo group (—N═N—) (e.g., III-48a), or combinations thereof. III-41a through III-48a and III-109a, as well as other illustrative, but non-limiting, aryls and heteroaryls as substrates are further described herein. Other non-hydrogen substituents are described herein.

The catalyst composition may be utilized to catalyze any suitable reaction under conversion conditions. Illustrative, but non-limiting, reactions that the catalyst composition may be used to catalyze include, but are not limited to: reductive detosylation; debenzylation; dephosphonation; degradation of a quaternary ammonium salt; hydrodefluorination; cross-coupling; a cyclization (e.g., a pinacol cyclization); cleavage of a sulfone; reductive alkylation; ester fragmentation; deoxygenation; ring opening; or combinations thereof. These aforementioned reactions in which the catalyst composition may be utilized to catalyze are further described below.

The catalyst composition may be utilized to catalyze, under conversion conditions, an at least partial reduction of a substrate, such as an at least partial reduction of an aryl or heteroaryl of a substrate, for example, substrates III-101a through III-109a, described below.

The catalyst composition may be utilized to catalyze, under conversion conditions, the reduction of bonds in a substrate, catalyze the breaking of bonds in a substrate, catalyze reactions involving a substrate, etc., or combinations thereof in the presence of light having a wavelength (or peak wavelength) in the visible range (e.g., about 350 nm to about 750 nm), or in a range from about 300 to about 800 nm, or from about 350 to 750 nm, or from about 350 to about 450 nm, or from about 375 to about 430 nm, such as from about 395 to about 415 nm, or about 405 nm.

The catalyst composition may be utilized to catalyze, under conversion conditions, the reduction of bonds in a substrate, catalyze the breaking of bonds in a substrate, catalyze reactions involving a substrate, etc., or combinations thereof in the presence of water.

### Conversion Processes

Aspects described herein also generally relate to conversion processes using the catalysts and catalyst compositions. A conversion process of the present disclosure may include: (a) forming a reaction mixture comprising a substrate and a catalyst composition; and (b) exposing the reaction mixture to light to convert the substrate, under conversion conditions, to a conversion product. Any suitable catalyst composition described herein may be utilized. For example, a suitable catalyst composition for the conversion process may include an organic photoredox catalyst described herein, such as an organic photoredox catalyst represented by formula (I) or formula (I-A), such as formula (I-A-1), such as Cat-1, Cat-2, Cat-3, Cat-4, or combinations thereof. The catalyst composition for the conversion process may further include any suitable reductant described herein. The catalyst composition for the conversion process may further include any suitable solvent or solvent system described herein.

Any suitable conversion conditions may be utilized at operation (b) of the process. Suitable conversion temperatures may include room temperature (from about 20° C. to about 25° C.), elevated temperatures (temperatures above room temperature), or combinations thereof. Suitable conversion temperatures may be in a range from about −10 to 100° C., such as from about 0 to about 80° C., such as from about 5 to about 70° C., such as from about 10 to about 60° C., such as from about 20 to about 50° C., such as from about 20 to about 40° C. Additionally, or alternatively, the conversion temperature may be greater than the freezing point of the solvent, less than the boiling point of the solvent, or combinations thereof. The conversion temperature is the temperature monitored by a temperature probe or an infrared (IR) probe. Conversion conditions at operation (b) of the process may include any suitable conversion pressure, such as about 500 kilopascals (kPa) or less, or in a range from about 80 to about 120 kPa, such as from about 90 to about 110 kPa, such as about 101 kPa (about 1 atm). Conversion conditions at operation (b) of the process may include any suitable conversion period, such as 200 hours (h) or less, such as about 100 h or less, such as about 80 h or less, such as about 50 h or less, such as about 24 hours or less, such as about 20 h or less, such as about 10 h or less, such as about 5 h or less, such as about 3 h or less, or in a range from about 1 to about 24 h, such as from about 1 to about 10 h, such as from about 1 to about 5 h, such as from about 2 to about 4 h. The conversion period is based on the period of exposure of the reaction mixture to the light.

The components of the reaction mixture at operation(s) (a) and/or (b) of the process may be mixed, stirred, or agitated by using suitable devices such as a mechanical stirrer, such as an overhead stirrer, a magnetic stirrer (for example, placing a magnetic stir bar in the vessel above a magnetic stirrer), a batch reaction vessel, a semi-batch reaction vessel, a continuous static mixer, or other suitable devices. For example, a stirrer having a blade or propeller may be rotated by receiving rotational power from a stirring motor to stir the one or more materials at suitable rotation speeds. Suitable rotation speeds may include from about 50 revolutions per minute (rpm) to about 1,500 rpm, such as from about 75 to about 1,000 rpm, such as from about 100 to about 900 rpm, such as from about 200 to about 800 rpm, such as from about 300 to about 700 rpm, such as from about 400 to about 600 rpm, such as from about 450 to about 550 rpm, such as about 500 rpm. Other rotation speeds are contemplated and may be selected based on the ability to mix the components sufficiently. Mechanical agitation or jet mixing may be used. Components of the reaction mixture at operation(s) (a) and/or (b) may be mixed, stirred, or agitated in the presence of a non-reactive gas, such as nitrogen gas (N₂), argon (Ar), or combinations thereof. For example, a non-reactive gas may be introduced with one or components of the reaction mixture to degas various components or otherwise remove unwanted gases such as oxygen from the reaction mixture.

Suitable molar ratios of the organic photoredox catalyst to the substrate in the reaction mixture may be in a range from about 1:10 to about 1:1,000, such as from about 1:20 to about 1:500, such as from about 1:30 to about 1:250, such as from about 1:40 to about 1:125, such as from about 1:50 to about 1:100, or in a range from about 1:10 to about 1:150, such as from about 1:25 to about 1:125, or from about 1:25 to about 1:75, such as about 1:50 (organic photoredox catalyst:substrate).

Suitable molar ratios of the substrate to the reductant in the reaction mixture may be in a range from about 1:0.5 to about 1:50, such as from about 1:1 to about 1:35, such as from about 1:2 to about 1:25, such as from about 1:5 to about 1:15, such as from about 1:8 to about 1:13, such as about 1:10 to about 1:12, such as about 1:11 (substrate:reductant)

Operation (b) of the process includes exposing the reaction mixture to light to convert the substrate to the conversion product. Operation (b) may be performed in any suitable reactor such as a photoreactor. Any suitable photoreactor may be utilized.

The photoreactor may be equipped with one or more light sources, such as from 1 to 10 light sources, such as from 1 to 6 light sources, such as from 1 to 4 light sources, such as from 1 to 2 light sources, such as 1 light source or 2 light sources. The light source may be any suitable light source such as a light emitting diode (LED), a lamp (such as a Kessil lamp), or combinations thereof. The light is sourced from the light source(s). Light sources may include those light sources emitting a wavelength of light (and/or a peak wavelength) in the visible range (e.g., about 350 nm to about 750 nm), or in a range from about 300 to about 800 nm, or from about 350 to 750 nm, or from about 350 to about 450 nm, or from about 375 to about 430 nm, such as from about 395 to about 415 nm, such as from about 400 nm to about 410 nm, or about 405 nm. In contrast to conventional technologies, conversion processes of the present disclosure may be performed using visible light.

By exposure of the reaction mixture to the light, the substrate is converted to a crude product mixture. The crude product mixture may include component(s) of the crude product mixture, reaction product(s) of two or more components of the crude product mixture, a remainder balance of remaining starting component(s), or combinations thereof. The crude product mixture includes the conversion product. The crude product mixture may optionally include unreacted starting materials, coproducts, byproducts, or combinations thereof. The crude product mixture may be subjected to purification techniques known in the art to obtain a purified form of the conversion product.

Any suitable substrate may be utilized in conversion processes described herein. Substrates may include aryls, heteroaryls, unsubstituted aryls, unsubstituted heteroaryls, substituted aryls, substituted heteroaryls, or combinations thereof.

“Aryl” and “aryl group” interchangeably refer to a hydrocarbon ring system comprising hydrogen, for example, 5 to 100 carbon atoms (such as 5 to 40, 6 to 30, or 6 to 14 carbon atoms), and at least one aromatic ring. The aryl may be monocyclic or polycyclic. The aryl may include spiro groups. The aryl may be unsubstituted or substituted by one or more substituents comprising an unsubstituted hydrocarbyl, a substituted hydrocarbyl, or a functional group comprising at least one element from Group 13-17 of the periodic table of the elements. Illustrative, but non-limiting, examples of unsubstituted hydrocarbyls, substituted hydrocarbyls, and functional groups comprising at least one element from Group 13-17 are described herein. When the aryl is unsubstituted, the aryl may be referred to as an unsubstituted aryl. When the aryl is substituted, the aryl may be referred to as a substituted aryl. Examples of aryls may include, but are not limited to benzene, naphthene, and fluorene. Examples of aryls substituted with a non-hydrogen substituent may include, but are not limited to, toluene, phenylmethyl (benzyl, Bn), 2-phenyl-ethyl, (3-chlorophenyl)-methyl, and 2-chloro-2-phenyl-eth-1-yl.

“Heteroaryl” and “heteroaryl group” interchangeably refer to a 5- to 100-membered ring system comprising hydrogen atoms, 1 to 99 carbon atoms, 1 or more heteroatoms, and at least one aromatic ring. For example, a heteroaryl may include a 5- to 14-membered ring system comprising hydrogen atoms, one to thirteen carbon atoms, one to six heteroatoms, and at least one aromatic ring. The heteroaryl may be monocyclic or polycyclic. The heteroaryl may include spiro groups. Heteroatoms of the heteroaryl may include nitrogen, oxygen, sulfur, phosphorous, or combinations thereof. Illustrative, but non-limiting, examples of unsubstituted hydrocarbyls, substituted hydrocarbyls, and functional groups comprising at least one element from Group 13-17 are described herein. When the heteroaryl is unsubstituted, the heteroaryl may be referred to as an unsubstituted heteroaryl. When the heteroaryl is substituted, the heteroaryl may be referred to as a substituted heteroaryl. Examples of heteroaryls may include, but are not limited to, furan, benzofuran, imidazole, indole, indazole, isoxazole, methylpyridine, oxazole, phenylpyridine, pyridine, pyrrole, pyrimidine, pyrazine, quinole, quinazole, quinoxaline, triazine, tetrazole, thiazole, thiene, benzothiophene, and triazole.

“Arene” refers to an aromatic hydrocarbon. “Heteroarene” refers to an aromatic hydrocarbon in which one of the carbons of the aromatic ring have been replaced by a heteroatom.

The substrate may include a substrate represented by formula (III-A):

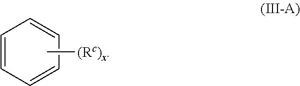

In formula (III-A), x is 0 or more. Each R^{C} of formula (III-A) may be, independently, a C1-C40 unsubstituted hydrocarbyl, a C1-C40 substituted hydrocarbyl, a functional group comprising at least one element from Group 13-17 of the periodic table of the elements, or two or more R^{C} join together to join together to form a monocyclic ring structure or a polycyclic ring structure. Suitable C1-C40 unsubstituted hydrocarbyls, C1-C40 substituted hydrocarbyls, and functional groups comprising at least one element from Group 13-17 of the periodic table of the elements for R^{C} of formula (III-A) may include those described herein, e.g., with respect to R^{A} of formula (I). When x is more than 1, each R^{C} of formula (III-A) may be the same or different. When a position on the aryl of formula (III-A) is not —R^{C} (e.g., when the substituent at the location is not —R^{C}) it is understood that hydrogen is located at that position.

In some aspects, which may be combined with other aspects, R^{C} of formula (III-A) may include a C1-C20 unsubstituted hydrocarbyl, such as a C1-C10 unsubstituted hydrocarbyl, such as: a C1-C10 alkyl or C1-C6 alkyl, such as methyl, ethyl, n-propyl, isopropyl, n-butyl, iso-butyl, sec-butyl, tert-butyl; a C2-C10 alkenyl or C2-C6 alkenyl, such as ethenylene (—CH═CH—), propenylene (—CH═CHCH₂—), 2-butenylene (—CH₂CH═CHCH₂—); a C2-C10 alkynyl or C2-C6 alkynyl, such as propargyl. In some aspects, which may be combined with other aspects, R^{C} of formula (III-A) may include a C1-C20 substituted hydrocarbyl, such as a C1-C6 substituted hydrocarbyl, wherein at least one hydrogen of the substituted hydrocarbyl is substituted with halogen, —COR*, —OR*, —C(O)R*, —C(O)NR*₂, —C(O)OR*, —NR*₂, —SR*, —PR*₂, —P(O)R*₂, —SO₂R*, —SO₃R*, wherein each R* is hydrogen or hydrocarbyl (such as unsubstituted hydrocarbyl or substituted hydrocarbyl), or wherein at least one hydrogen of the substituted hydrocarbyl is substituted with —OH, —F, —Cl, —Br, —I, —NH₂, —NO₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

Illustrative, but non-limiting, examples of suitable substrates useful in conversion processes described herein may include one or more of the following (Me is methyl):

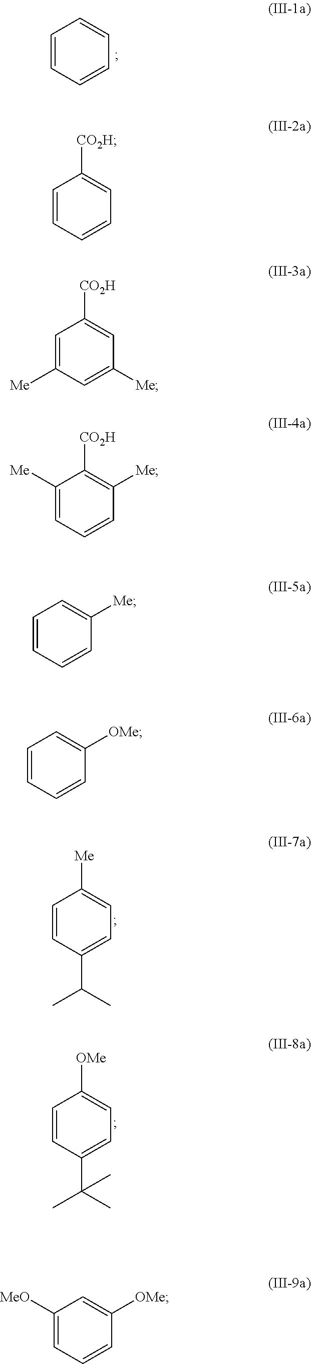

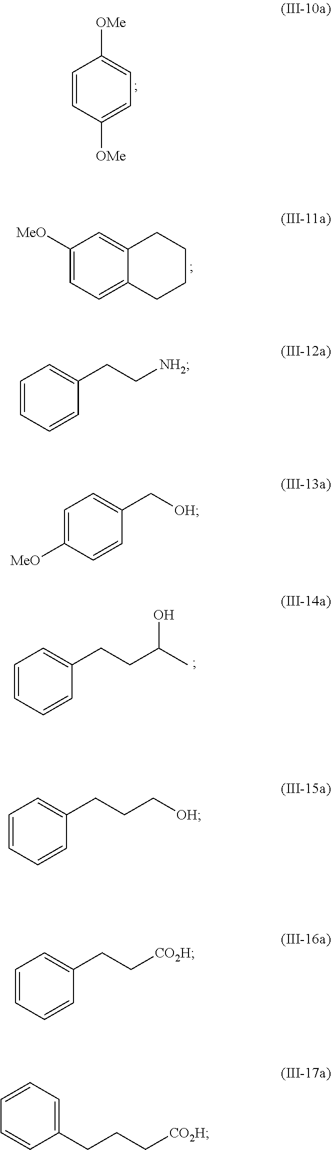

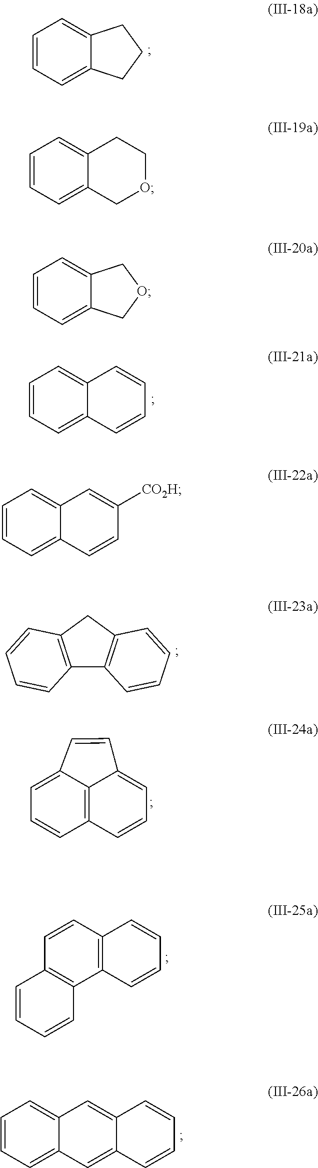

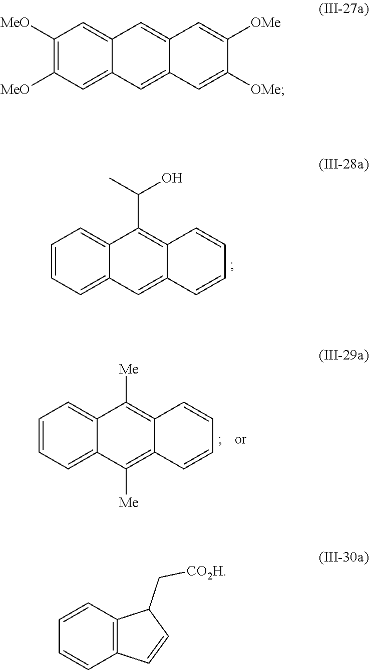

Conversion processes described herein may be utilized to convert these substrates to one or more of the following corresponding conversion products (Me is methyl):

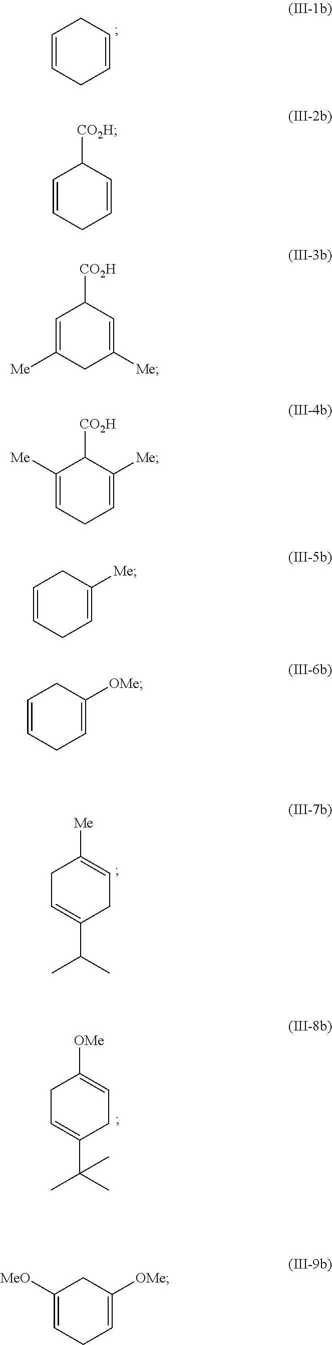

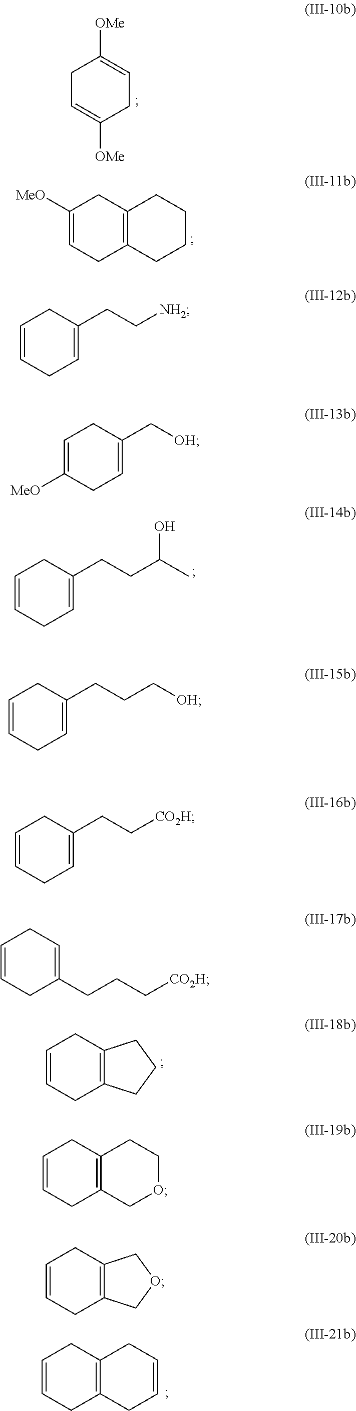

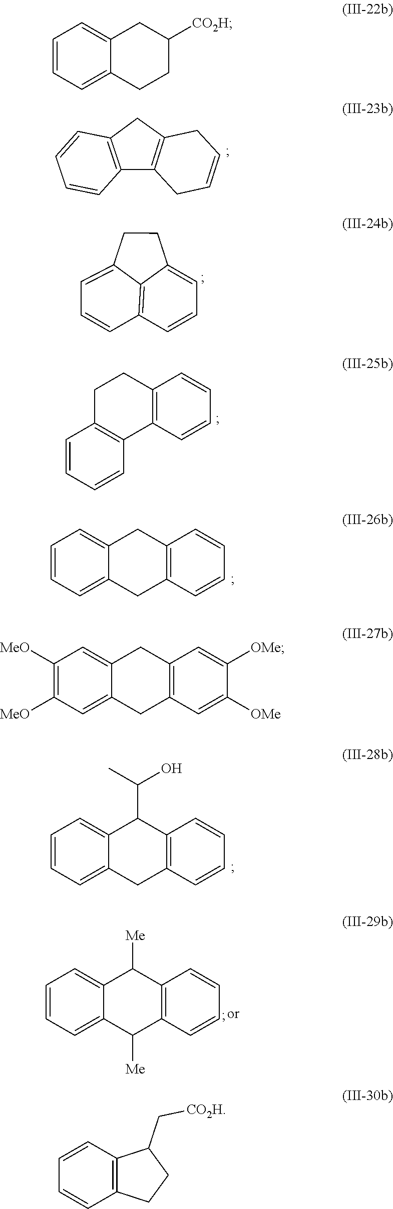

As shown, the reduction typically does not reduce the aromatic system all the way, for example, to fully reduced species such as cycloalkyl and heterocycloalkyl species.

As used herein, a substrate may be specified with an “a” after the number, while the conversion product includes “b” or a “c” after the number unless specified to the contrary or the context clearly indicates otherwise. For example, a substrate and a corresponding conversion product are III-1a and III-1b, respectively. As another example, a substrate and two corresponding conversion products are substrate III-53a and conversion products III-53b and III-53c.

Illustrative, but non-limiting, examples of substrates useful in conversion processes described herein may include one or more of the following substrates (Me is methyl, Ph is phenyl):

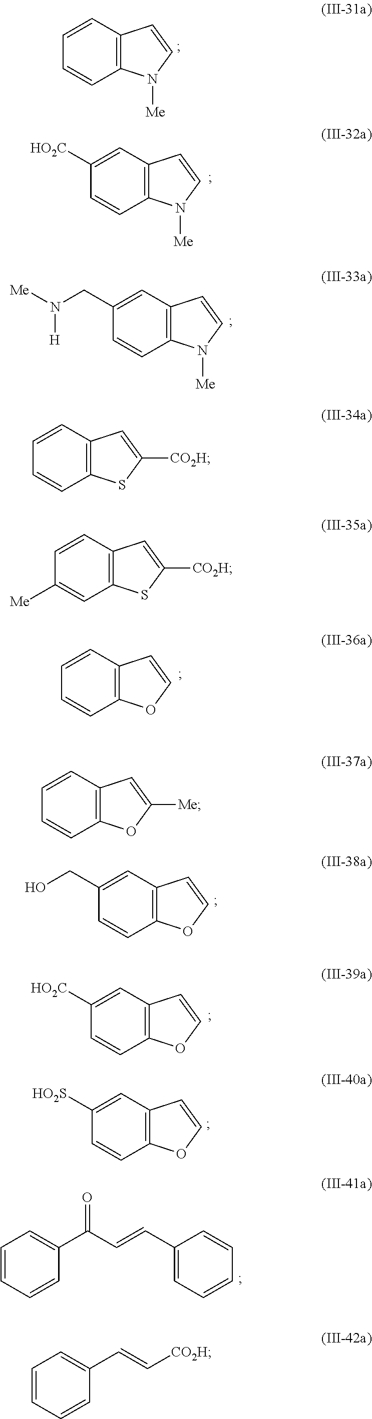

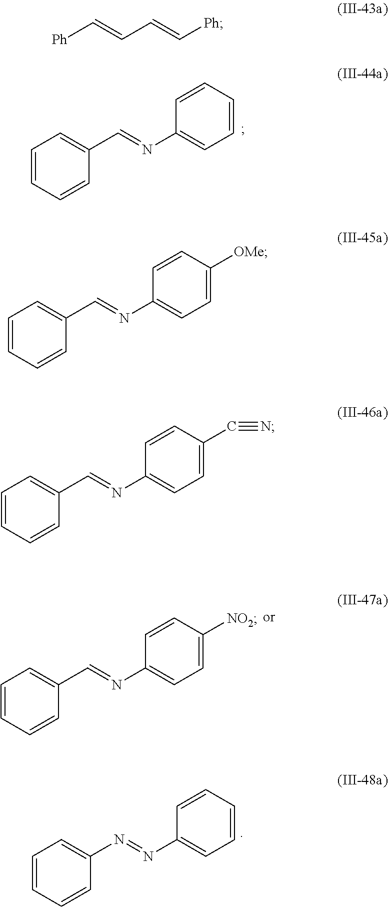

Conversion processes described herein may be utilized to convert these substrates to one or more of the following corresponding conversion products (Me is methyl):

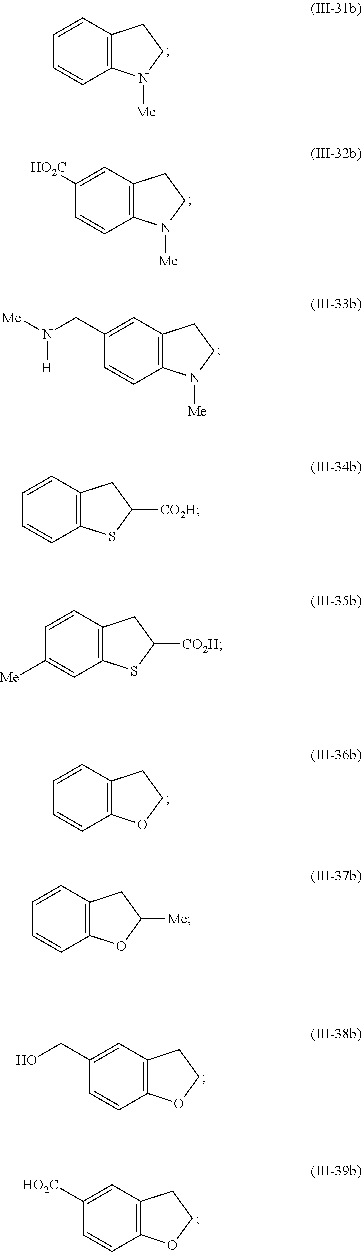

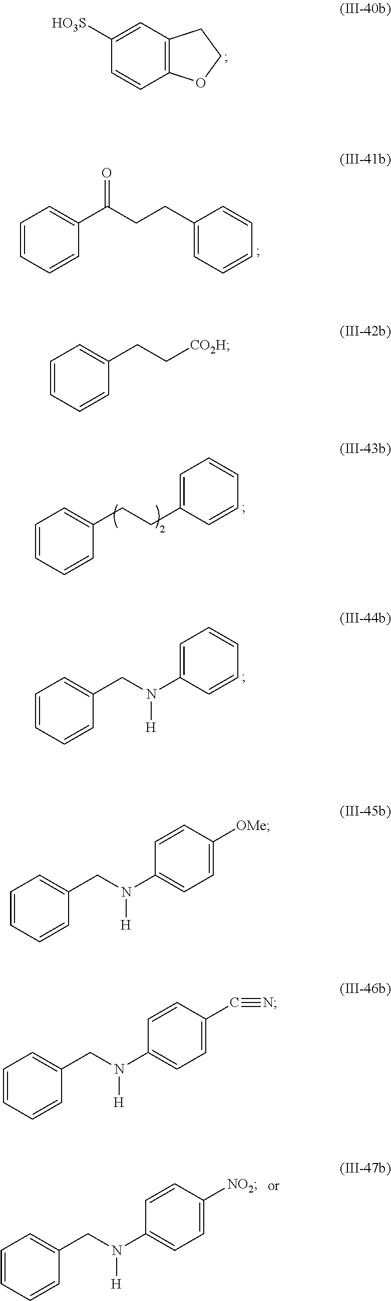

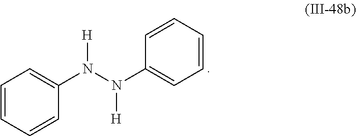 [SMILES: c1ccc(NNc2ccccc2)cc1]

Illustrative, but non-limiting, examples of substrates useful in conversion processes described herein may include one or more of the following substrates (Me is methyl, tBu is tert-butyl, Ph is phenyl):

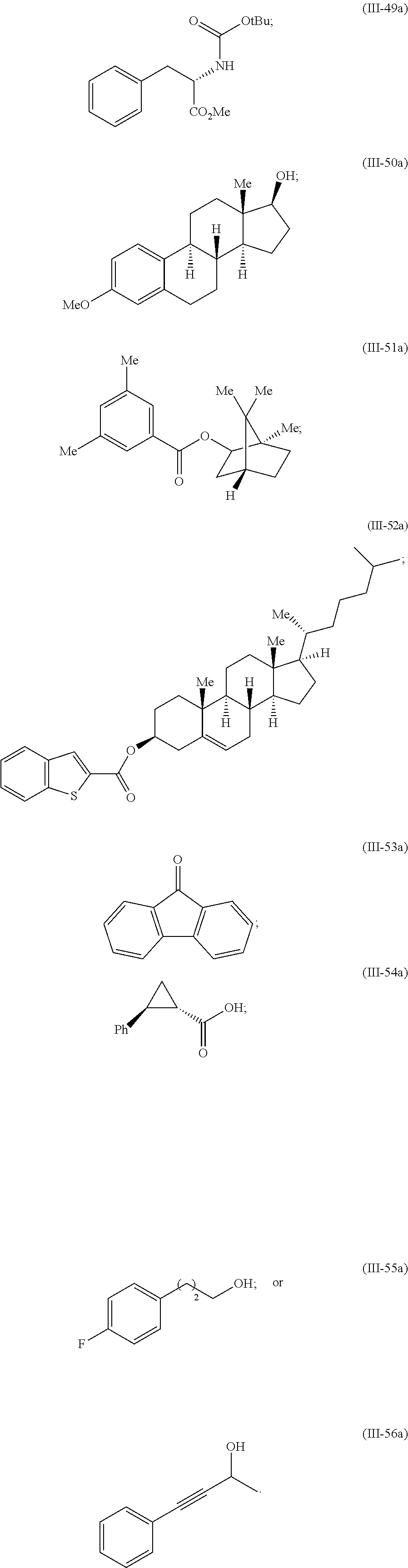

Conversion processes described herein may be utilized to convert these substrates to one or more of the following corresponding conversion products (Me is methyl, tBu is tert-butyl, Ph is phenyl):

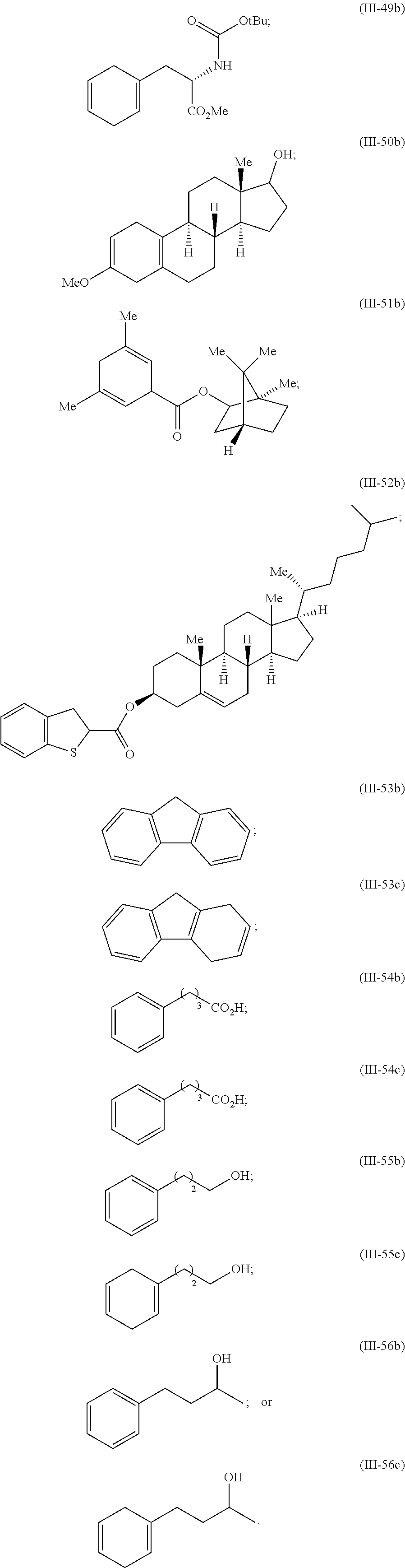

Conversion products III-53c, III-54c, III-55c, and III-56c, represent two reductions of the corresponding substrates III-53a, III-54a, III-55a, and III-56a, respectively. For example, conversion product III-53b is the deoxygenation product from substrate III-53a, and conversion product III-53c is the deoxygenation and arene reduction product from substrate III-53a. The selectivity of conversion processes described herein may be controlled to select one or both of the conversion products by, for example, the molar ratio of the organic photoredox catalyst to the substrate, the molar ratio of the substrate to the reductant, the conversion period, or combinations thereof.

Conversion processes of the present disclosure may be utilized to at least partially reduce an aryl or heteroaryl group of a substrate, for example, at least partially reduce an aryl or heteroaryl group of one or more of the following substrates (Me is methyl):

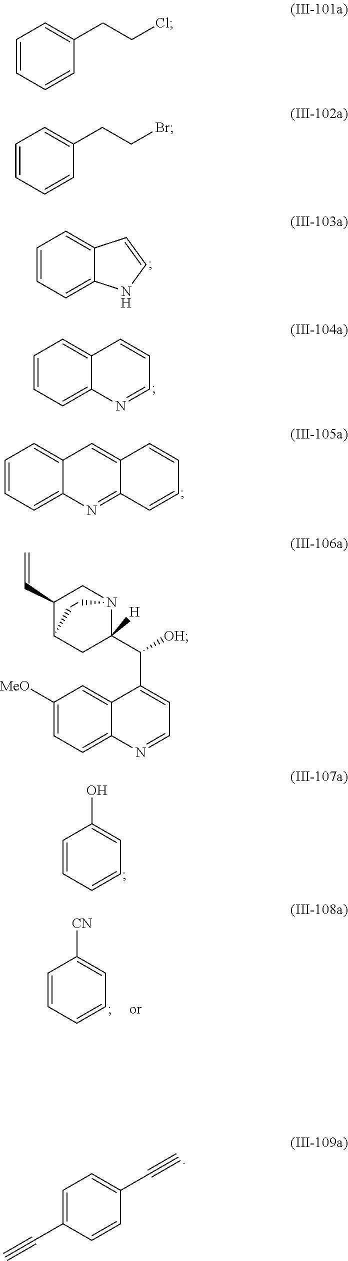

Illustrative, but non-limiting, examples of conversion products of the at least partial reduction of substrates III-101a through III-108a may include (Me is methyl):

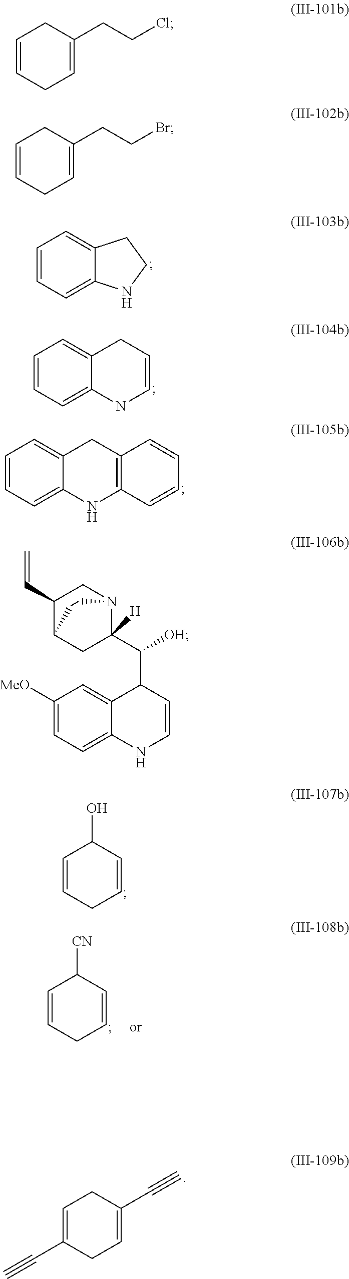

Conversion processes of the present disclosure may be utilized to selectively reduce substrates. For example, substrates having both (a) an aryl group or heteroaryl group; and (b) an alkynyl group, alkenyl group, an imine group (R**—C═N—R**), or an azo group (R**—N═N—R**) chemically bonded to the aryl group or heteroaryl group may be submitted to conversion processes described herein to selectively reduce the alkynyl group, alkenyl group, imine group, or azo group. With respect to the imine group and azo group, at least one of R** refers to the aryl group or heteroaryl group. The conversion process may be optionally performed to reduce both (a) the aryl group or heteroaryl group and (b) the alkynyl group, alkenyl group, imine group, or the azo group. The reduction of the aryl group or heteroaryl group may be an at least partial reduction of the aryl group or heteroaryl group. The reduction of the aryl group or heteroaryl group may be performed after the selective reduction of the alkynyl group, alkenyl group, imine group, or the azo group to which it is coupled. Illustrative, but non-limiting, examples of the selective reduction and reduction of both (a) the aryl group or heteroaryl group and (b) the alkynyl group, alkenyl group, imine group, or the azo group may include, but are not limited to, one or more of the following:

(1) The reduction of substrate III-56a to conversion product III-56b (selective reduction of the alkynyl group) and conversion product III-56c (reduction of the aryl group).

(2) The reduction of substrate III-43a to conversion product III-43b (selective reduction of the alkenyl group).

(3) The reduction of substrates III-44a, III-45a, III-46a, and III-47a to conversion products III-44b, III-45b, III-46b, and III-47b, respectively (selective reduction of the imine group).

(4) The reduction of substrate III-48a to conversion product III-48b (selective reduction of the azo group).

Conversion processes of the present disclosure may be utilized to remove functional groups from an aryl or a heteroaryl (e.g., break chemical bonds). For example, de-alkoxylations, de-phosphonations, and de-sulfonations may be performed utilizing aspects described herein to form the corresponding aromatic ring or heteroaromatic ring as shown with a substrate represented by formula (III-B) and the corresponding conversion product represented by formula (III-B1).

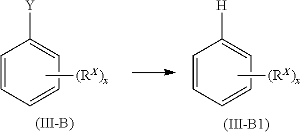

In formula (III-B), each R^{X} may be, independently, a C1-C20 unsubstituted hydrocarbyl, a C1-C20 substituted hydrocarbyl, a functional group comprising at least one element from Group 13-17 of the periodic of the elements, or two or more R^{X} join together to join together to form a monocyclic ring structure or a polycyclic ring structure; and x may be an integer from 0 to 5, such as from 0 to 2, such as 0 or 1. When x is 2 or more, each R^{X} of formula (III-B) may be the same or different. When x of formula (III-B) is less than 5, the remaining substituents on the individual aryl moieties of (III-B) are hydrogen. Suitable C1-C20 unsubstituted hydrocarbyls, C1-C20 substituted hydrocarbyls, and functional groups comprising at least one element from Group 13-17 of the periodic table of the elements for R^{X} of formula (III-B) may include those described herein, e.g., with respect to R^{A} of formula (I) and/or with respect to R^{C} of formula (III-A).

In formula (III-B), Y may include: an alkoxyl (—OR*); a phosphonyl (—P(O)(OR*)₂; or a sulfonyl (—S(O)₂R″), wherein R* may be, independently, a hydrogen or a hydrocarbyl, such as a C1-C20 unsubstituted hydrocarbyl or C1-C20 substituted hydrocarbyl, for example, those unsubstituted and substituted hydrocarbyls described herein with respect to R^{A} of formula (I) and R^{C} of formula (III-A), and wherein R″ with respect to the sulfonyl may include an aryl, a heteroaryl, a hydrogen, or a hydrocarbyl, such as a C1-C20 unsubstituted hydrocarbyl or C1-C20 substituted hydrocarbyl, for example, those unsubstituted and substituted hydrocarbyls described herein with respect to R^{A} of formula (I) and R^{C} of formula (III-A). Illustrative, but non-limiting examples, of alkoxyls for Y of formula (III-B) may include —O—CH₂Ph (benzyl alkoxyl), -OiPr (isopropyl alkoxyl), or -OtBu (tert-butyl alkoxyl). Illustrative, but non-limiting examples, of phosphonyls for Y of formula (III-B) may include —P(O)(OEt)₂ or —P(O)(OMe)₂. Illustrative, but non-limiting examples, of sulfonyls for Y of formula (III-B) may include —S(O)₂(Ph) or —S(O)₂(CH2C(O)(OMe)).

In formula (III-B1), H is hydrogen. Each of R^{X} and x of formula (III-B1) corresponds to R^{X} and x of formula (III-B). When a position on the aryl moiety of formula (III-B1) is not —R^{X}, it is understood that hydrogen is located at that position.

Illustrative, but non-limiting, examples of substrates represented by formula (III-B) may include one or more of the following:

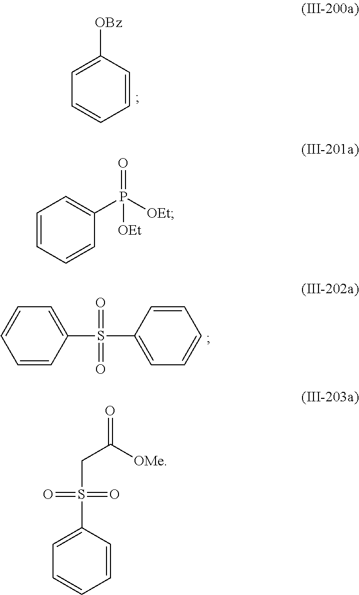

Conversion processes described herein may be used for various reactions, including, but not limited to: detosylation of a substrate such as an indole; degradation of a substrate such as a quaternary ammonium salt functional group chemically bonded to an aryl; hydrodefluorination of a substrate such as an aryl; conversion of an arylfluoride to a coupling product such as a boron-containing coupling product; a cyclization of a substrate such as a di aryl dialdehyde or a diaryl; reductive alkylation of a substrate such as benzoic acid; aryl ester fragmentation; or combinations thereof.

With respect to detosylation of an indole substrate, any suitable indole substrate represented by formula (III-C) may be utilized (Ts is —SO₂—C₆H₄—CH₃):

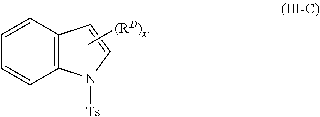

In formula (III-C), each R^{D} may be, independently, a C1-C20 unsubstituted hydrocarbyl, a C1-C20 substituted hydrocarbyl, a functional group comprising at least one element from Group 13-17 of the periodic of the elements, or two or more R^{D} join together to join together to form a monocyclic ring structure or a polycyclic ring structure; and x may be 0 or more, such as an integer from 0 to 6, such as from 0 to 3, such as from 0 to 2. When x is more than 1, each R^{D} of formula (III-C) may be the same or different. Suitable C1-C20 unsubstituted hydrocarbyls, C1-C20 substituted hydrocarbyls, and functional groups comprising at least one element from Group 13-17 of the periodic table of the elements for R^{D} of formula (III-C) may include those described herein, e.g., with respect to R^{A} of formula (I) and/or with respect to R^{C} of formula (III-A)

The indole of formula (III-C) may be free of non-hydrogen substituents, in which case x of formula (III-C) is 0. Alternatively, when x of formula (III-C) is 1 or more, the indole is substituted with one or more non-hydrogen substituents, for example, the C1-C20 unsubstituted hydrocarbyl, the C1-C20 substituted hydrocarbyl, or the functional group comprising at least one element from Group 13-17 of the periodic of the elements. When a position on the indole moiety of formula (III-C) is not —R^{D} (e.g., when the substituent at the location is not —R^{D}) it is understood that hydrogen is located at that position.

Conversion products formed from detosylation may include those conversion products represented by formula (III-C1):

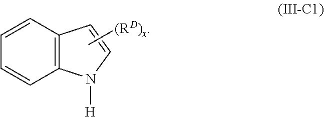

Each of R^{D} and x of formula (III-C1) corresponds to R^{D} and x of formula (III-C). As shown, the conversion product of formula (III-C1) is detosylated, being free of the toluenesulfonyl group (also referred to as tosyl group or “Ts”). When a position on the indole moiety of formula (III-C1) is not —R^{D} (e.g., when the substituent at the location is not —R^{D}) it is understood that hydrogen is located at that position. For example, when x of formula (III-C1) is 2, the remaining four substituents on the indole moiety of (III-C1) are hydrogen.

Illustrative, but non-limiting, examples of indoles as substrates for detosylation in conversion processes described herein may include substrate III-57a and substrate III-58a (Me is methyl and Ts is —SO₂—C₆H₄—CH₃):

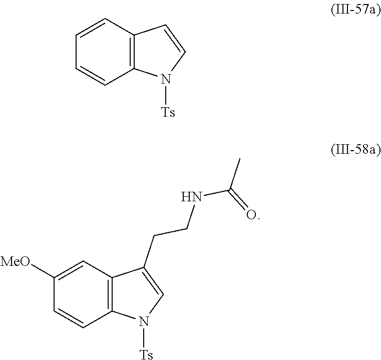 [SMILES: Cc1ccc(S(=O)(=O)n2ccc3ccccc32)cc1 ; COc1ccc2c(c1)c(CCNC(C)=O)cn2S(=O)(=O)c1ccc(C)cc1]

Substrate III-57a is a tosylated indole that does not include non-hydrogen substituents. Substrate III-58a is a tosylated indole that includes two substituted hydrocarbyl substituents. Corresponding conversion products from substrates III-57a and III-58a are detosylated conversion products III-57b and III-58b:

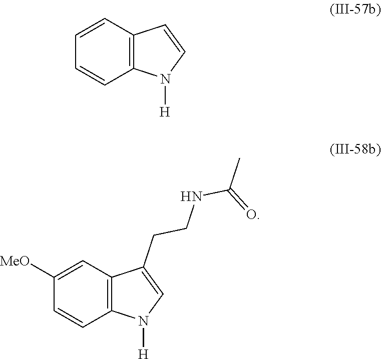 [SMILES: c1ccc2[nH]ccc2c1 ; COc1ccc2[nH]cc(CCNC(C)=O)c2c1]

With respect to degradation of a quaternary ammonium salt moiety chemically bonded to an aryl, any suitable substrate represented by formula (III-D) may be used:

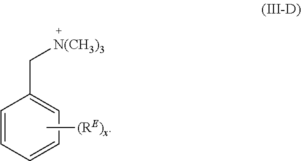

In formula (III-D), each R^{E} may be, independently, a C1-C20 unsubstituted hydrocarbyl, a C1-C20 substituted hydrocarbyl, a functional group comprising at least one element from Group 13-17 of the periodic table of the elements, or two or more R^{E} join together to join together to form a monocyclic ring structure or a polycyclic ring structure; and x may be 0 or more, such as an integer from 0 to 5, such as from 0 to 3, such as from 0 to 2. When x is 2 or more, each R^{E} of formula (III-D) may be the same or different. When a position on the aryl moiety of formula (III-D) is not —R^{E} or —CH₂Me (e.g., when the substituent at the location on the aryl moiety is not —R^{E} or —CH₂Me) it is understood that hydrogen is located at that position. Suitable C1-C20 unsubstituted hydrocarbyls, C1-C20 substituted hydrocarbyls, and functional groups comprising at least one element from Group 13-17 of the periodic table of the elements for R^{E} of formula (III-D) may include those described herein, e.g., with respect to R^{A} of formula (I) and/or R^{C} of formula (III-A).

Conversion products formed from the degradation of the substrate represented by formula (III-D) may include those conversion products represented by formula (III-D1):

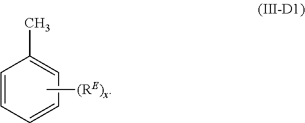

Each of R^{E} and x of formula (III-D1) corresponds to R^{E} and x of formula (III-D). As shown, the conversion product of formula (III-D1) is degraded, being free of the ammonium group. When a position on the aryl moiety of formula (III-D1) is not —R^{E} or —CH₃ (e.g., when the substituent at the location on the aryl moiety is not —R^{E} or —CH₃) it is understood that hydrogen is located at that position. For example, in formula (III-D1), when x is 1, the remaining four substituents on the aryl moiety of (III-D1) are hydrogen.

An illustrative, but non-limiting, example of a suitable substrate for degradation of a quaternary ammonium salt functional group chemically bonded to an aryl may include substrate III-71a with the corresponding conversion product from the degradation including conversion product III-71b (Me is methyl):

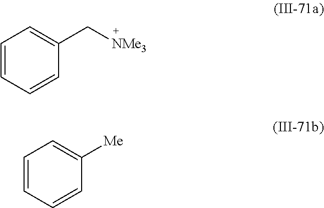

Conversion processes of the present disclosure may be utilized to cyclize, e.g., a diaryl dialdehyde, a diaryl diketone, or a mixed diaryl monoaldehyde-monoketone. Such cyclizations may include Pinacol cyclizations. Generally, Pinacol cyclization is a reaction in which a carbon-carbon bond is formed between two carbonyl groups, for example, two aldehydes, two ketones, or an aldehyde and a ketone. Any suitable substrate may be utilized for such cyclizations, e.g., those substrates represented by formula (III-E):

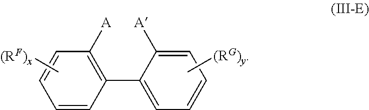

In formula (III-E), each of A and A′ may be, independently, an aldehyde or a ketone; each R^{F} and R^{G} may be, independently, a C1-C20 unsubstituted hydrocarbyl, a C1-C20 substituted hydrocarbyl, a functional group comprising at least one element from Group 13-17 of the periodic of the elements, or two or more R^{F} or two or more R^{G} join together to join together to form a monocyclic ring structure or a polycyclic ring structure; and each of x and y may be, independently, an integer from 0 to 4, such as from 0 to 2, such as 0 or 1. When x is 2 or more, each R^{F} of formula (III-E) may be the same or different. When y is 2 or more, each R^{G} of formula (III-E) may be the same or different. When x is less than 4 and/or when y is less than 4, the remaining substituents on the individual aryl moieties of formula (III-E) are hydrogen. Suitable C1-C20 unsubstituted hydrocarbyls, C1-C20 substituted hydrocarbyls, and functional groups comprising at least one element from Group 13-17 of the periodic table of the elements for R^{F} and R^{G} of formula (III-E) may include those described herein, e.g., with respect to R^{A} of formula (I) and/or R^{C} of formula (III-A).

Conversion products formed from the cyclization may include those conversion products represented by formula (III-E1):

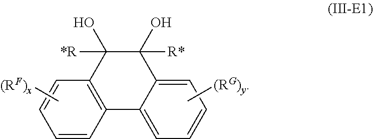

In formula (III-E1), each of R^{F}, R^{G}, x, and y correspond to R^{F}, R^{G}, x, and y of formula (III-E). Each R* of formula (III-E1) may be, independently, a hydrogen or a hydrocarbyl depending on, e.g., whether A and A′ of substrate (III-E) are aldehydes or ketones. In formula (III-E1), when x is less than 4 and/or when y is less than 4, the remaining substituents on the individual aryl moieties (III-E1) are hydrogen.

Illustrative, but non-limiting, examples of suitable substrates for the cyclization may include dialdehyde substrate III-63a, diketone substrate III-210a, and mixed aldehyde ketone substrate III-211a. Corresponding products from the cyclization may include conversion products III-63b, III-210b, and III-21 1b.

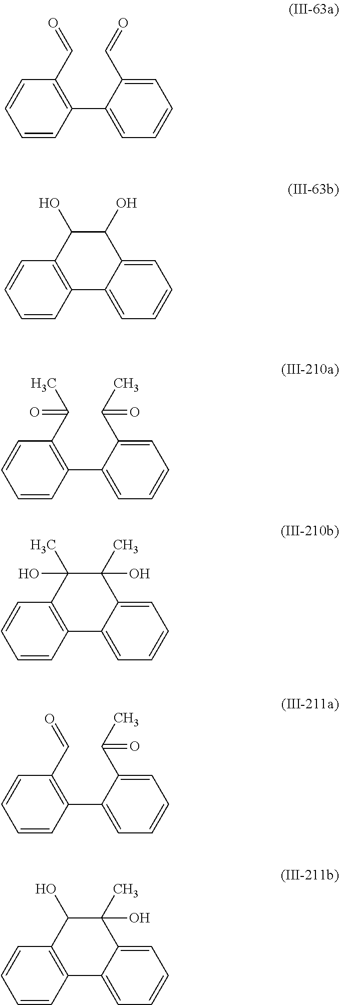

With respect to cross-couplings, conversion processes described herein may be utilized to convert an organofluoride to a coupling product in a cross-coupling reaction with, e.g., a boron-containing reactant. Organofluoride substrates may include those substrates represented by formula (III-F):

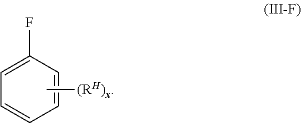

In formula (III-F), each R^{H} may be, independently, a C1-C20 unsubstituted hydrocarbyl, a C1-C20 substituted hydrocarbyl, a functional group comprising at least one element from Group 13-17 of the periodic table of the elements, or two or more R^{H} join together to join together to form a monocyclic ring structure or a polycyclic ring structure; and x of formula (III-F) may be, independently, 0 or more, such as an integer from 0 to 5, such as from 0 to 3, such as from 0 to 2. When x is 2 or more, each R^{H} of formula (III-F) may be the same or different. When a position on the aryl moiety of formula (III-F) is not —R^{H} or —F (e.g., when the substituent at the location on the aryl moiety is not —R^{H} or —F) it is understood that hydrogen is located at that position. Suitable C1-C20 unsubstituted hydrocarbyls, C1-C20 substituted hydrocarbyls, and functional groups comprising at least one element from Group 13-17 of the periodic table of the elements for R^{H} of formula (III-F) may include those described herein, e.g., with respect to R^{A} of formula (I) and/or R^{C} of formula (III-A).

Conversion products formed from the conversion of organofluoride substrates represented by formula (III-F) by the cross-coupling reaction may be represented by formula (III-F1):

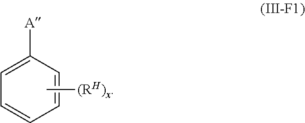

A″ of formula (III-F1) may include a boron-containing functional group. In formula (III-F1), each of R^{H} and x correspond to R^{H} and x of formula (III-F). When a substituent at a position on the aryl moiety of formula (III-F1) is not —R^{H} or -A″, it is understood that hydrogen is located at that position.

For the cross-coupling, a coupling partner (A*), such as a boron-containing reactant, is utilized. The cross-coupling may be referred to as a borylation reaction. Boron-containing reactants useful as coupling partners A* may include, but are not limited to, bis(pinacolato)diboron (B₂Pin₂), bis(2,4-dimethylpentane-2,4-glycolato)diboron, bis(neopentyl glycolato)diboron (B₂(neop)₂), or combinations thereof. For example, coupling partners A* and the corresponding A″ of formula (III-F1) may include those shown below. A″ of formula (III-F1) is a boron-containing group in the coupling product. The wavy bond represents the connection to the aryl group of formula (III-F1).

### Coupling Partners (a*)

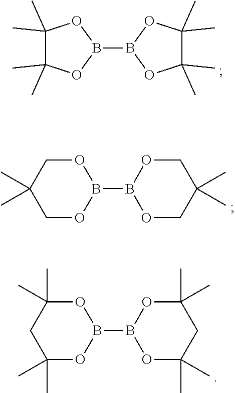

### Boron-Containing Group in the Coupling Product (A″)

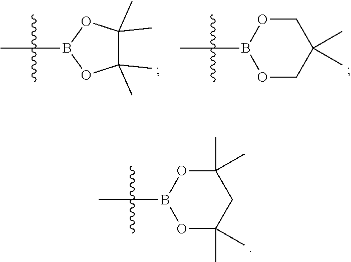

Illustrative, but non-limiting, examples of suitable substrates for cross-coupling of the organofluoride to the coupling product (e.g., the borylation reaction) may include organofluoride substrates III-61a and III-62a. Corresponding products from the cyclization may include conversion products III-61b and III-62b, respectively.

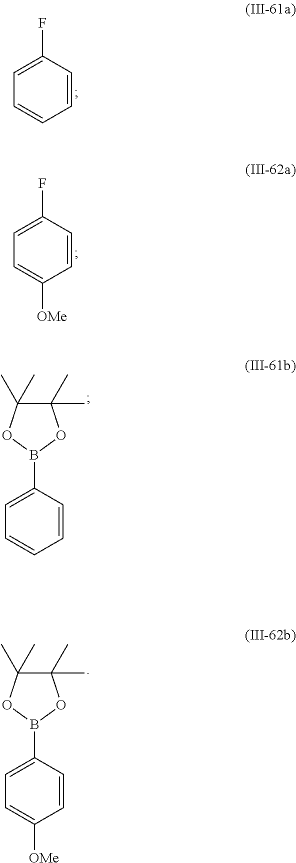

Conversion processes of the present disclosure may be used for reductive alkylation reactions, e.g., reductive alkylation of an aryl acid. Any suitable aryl acid substrate may be utilized for reductive alkylation, such as those substrates represented by (III-G):

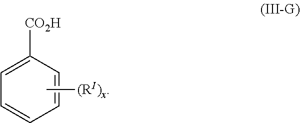

In formula (III-G), each R^{I} may be, independently, a C1-C20 unsubstituted hydrocarbyl, a C1-C20 substituted hydrocarbyl, a functional group comprising at least one element from Group 13-17 of the periodic table of the elements, or two or more R^{I} join together to join together to form a monocyclic ring structure or a polycyclic ring structure; and x may be, independently, 0 or more, such as an integer from 0 to 5, such as from 0 to 3, such as from 0 to 2. When x is 2 or more, each R^{I} of formula (III-G) may be the same or different. When a substituent at a position on the aryl moiety of formula (III-G) is not —R or —CO₂H, it is understood that hydrogen is located at that position.

Suitable C1-C20 unsubstituted hydrocarbyls, C1-C20 substituted hydrocarbyls, and functional groups comprising at least one element from Group 13-17 of the periodic table of the elements for R^{I} of formula (III-G) may include those described herein, e.g., with respect to R^{A} of formula (I) and/or R^{C} of formula (III-A).

Conversion products from the reductive alkylation may include those conversion products represented by formula (III-G1):

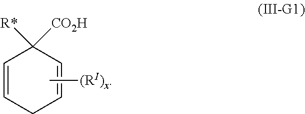

In formula (III-G1), each of R^{I} and x correspond to R^{I} and x of formula (III-G). R* of formula (III-G1) may be, independently, a C1-C20 unsubstituted hydrocarbyl, such as a C1-C6 unsubstituted hydrocarbyl, such as methyl, ethyl, propyl, butyl, and the like. For the reductive alkylation, any suitable alkylating reagent may be utilized, for example, an alkyl halide, such as an alkyl iodide, for example, methyl iodide, ethyl iodide, etc. Hydrogen atoms are implied in the conversion product represented by formula (III-G1), e.g., when x is less than 5.

An illustrative, but non-limiting, example of a suitable substrate for the reductive alkylation may include benzoic acid (III-64a). The corresponding product from the reductive alkylation may include conversion product III-64b.

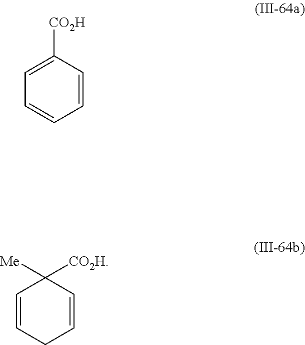 [SMILES: O=C(O)c1ccccc1 ; CC1(C(=O)O)C=CCC=C1]

Conversion processes described herein may include fragmentation of aryl esters. Any suitable aryl ester substrate may be utilized for the fragmentation, such as those substrates represented by (III-H):

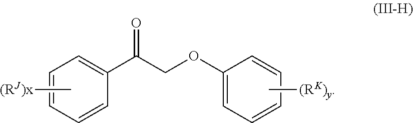

In formula (III-H), each of R^{J} and R^{K} may be, independently, a C1-C20 unsubstituted hydrocarbyl, a C1-C20 substituted hydrocarbyl, a functional group comprising at least one element from Group 13-17 of the periodic of the elements, or two or more R¹ or two or more R^{K} join together to join together to form a monocyclic ring structure or a polycyclic ring structure; and each of x and y may be, independently, an integer from 0 to 5, such as from 0 to 2, such as 0 or 1. When x is 2 or more, each R^{J} of formula (III-H) may be the same or different. When y is 2 or more, each R^{K} of formula (III-H) may be the same or different. When x of formula (III-H) is less than 5 and/or when y of formula (III-H) is less than 5, the remaining substituents on the individual aryl moieties of (III-H) are hydrogen. Suitable C1-C20 unsubstituted hydrocarbyls, C1-C20 substituted hydrocarbyls, and functional groups comprising at least one element from Group 13-17 of the periodic table of the elements for R^{J} and R^{K} of formula (III-H) may include those described herein, e.g., with respect to R^{A} of formula (I) and/or R^{C} of formula (III-A).

Conversion products formed from the ester fragmentation may include those conversion products represented by formula (III-H1) and/or formula (III-H2):

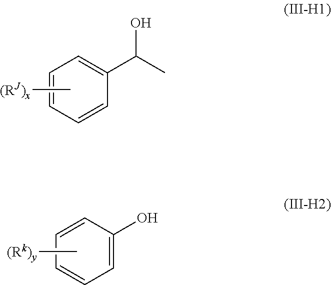

In formula (III-H1) and formula (III-H2), each of R^{J}, R^{K}, x, and y correspond to R^{J}, R^{K}, x, and y of formula (III-H). Hydrogen atoms are implied in the conversion products represented by formula (III-H1) and formula (III-H2), e.g., when x is less than 5 and/or when y is less than 5.

An illustrative, but non-limiting, example of a suitable substrate for the ester fragmentation may include substrate III-65a, and the corresponding products from the ester fragmentation may include conversion products III-65b and/or III-65c.

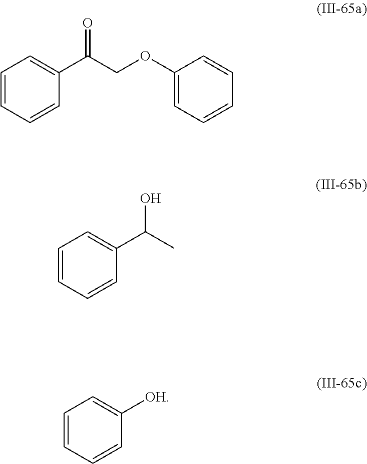

As shown, the ester fragmentation may form two alcohol conversion products, e.g., II-65b and III-65c. Such ester fragmentations (for example, aryl ester fragmentations) may be utilized for various applications. For example, the ester fragmentation may be used to decompose ester groups present in biomass to alcohol conversion products. As another example, synthetically-modified lignin that has new ester bonds relative to natural lignin may be decomposed using the ester fragmentation. As another example, in plant cell walls, lignin-carbohydrate complexes—linking lignin subunits to sugar residues—may be decomposed using the ester fragmentation. For biomass, lignin, and synthetically modified lignin, among other suitable substrates, the aryl moieties may be substituted with, for example, —OH groups, alkoxyl groups, or combinations thereof.

### Hydrodefluorination Processes

Hydrodefluorination is another conversion process in which catalysts and catalyst compositions of the present disclosure may be utilized. A hydrodefluorination process of the present disclosure may include: (a) forming a reaction mixture comprising a fluorine-containing substrate and a catalyst composition; and (b) exposing the reaction mixture to light to convert the substrate, under conversion conditions, to a conversion product. Operation (a) and (b) of the hydrodefluorination process may be performed as described herein with respect to other conversion processes.

Any suitable catalyst composition described herein may be utilized for the hydrodefluorination process. For example, a suitable catalyst composition for the hydrodefluorination process may include an organic photoredox catalyst described herein, such as an organic photoredox catalyst represented by formula (I) or formula (I-A), such as formula (I-A-1), such as Cat-1, Cat-2, Cat-3, Cat-4, or combinations thereof. The catalyst composition for the hydrodefluorination process may further include any suitable reductant described herein. The catalyst composition for the hydrodefluorination process may further include any suitable solvent or solvent system described herein. Conversion conditions, molar ratios, etc. described herein with respect to other conversion processes may be utilized for the hydrodefluorination process. For example, conversion temperatures for the hydrodefluorination process may be in a range from about −10 to 100° C., conversion pressures may be about 500 kPa or less, and conversion periods may be about 200 h or less, such as those temperatures, pressures, and periods described herein with respect to other conversion processes. Molar ratios of the organic photoredox catalyst to the substrate in the reaction mixture may be in a range from about 1:10 to about 1:1,000 (organic photoredox catalyst:substrate) and molar ratios of the substrate to the reductant in the reaction mixture may be in a range from about 1:0.5 to about 1:50 (substrate:reductant), such as those molar ratios described herein with respect to other conversion processes. Suitable rotation speeds for mixing or stirring may be in a range from about 50 rpm to about 1,500 rpm, such as those described herein with respect to other conversion processes. Light sources utilized may include those light sources emitting a wavelength of light (and/or a peak wavelength) in the visible range, or in a range from about 300 to about 800 nm, such as those ranges described herein with respect to other conversion processes. Operation (b) of the hydrodefluorination process may be performed in any suitable reactor such as a photoreactor.

By exposure of the reaction mixture to the light, the substrate for the hydrodefluorination process is converted to a crude product mixture. The crude product mixture may include component(s) of the crude product mixture, reaction product(s) of two or more components of the crude product mixture, a remainder balance of remaining starting component(s), or combinations thereof. The crude product mixture includes the conversion product. The crude product mixture may optionally include unreacted starting materials, coproducts, byproducts, or combinations thereof. The crude product mixture may be subjected to purification techniques known in the art to obtain a purified form of the conversion product.

Fluorine-containing substrates for the hydrodefluorination process may include those substrates represented by formula (III-I):

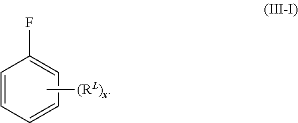

In formula (III-I), F is fluorine. R^{L} of formula (III-I) may be, independently, a C1-C20 unsubstituted hydrocarbyl, a C1-C20 substituted hydrocarbyl, a functional group comprising at least one element from Group 13-17 of the periodic of the elements, or two or more R^{L} join together to join together to form a monocyclic ring structure or a polycyclic ring structure; and x may be an integer from 0 to 5, such as from 0 to 2, such as 0 or 1. When x is 2 or more, each R^{L} of formula (III-I) may be the same or different. When a substituent at a position on the aryl moiety of formula (III-I) is not —R^{L} or —F, it is understood that hydrogen is located at that position. Suitable C1-C20 unsubstituted hydrocarbyls, C1-C20 substituted hydrocarbyls, and functional groups comprising at least one element from Group 13-17 of the periodic table of the elements for R^{L} of formula (III-I) may include those described herein, e.g., with respect to R^{A} of formula (I) and/or R^{C} of formula (III-A).

Generally, the hydrodefluorination reaction replaces a carbon-fluorine bond with a carbon-hydrogen bond. Accordingly, conversion products from the hydrodefluorination process may include those conversion products represented by formula (III-I1):

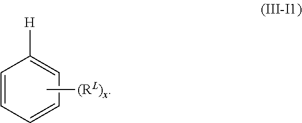

In formula (III-I1), H is hydrogen. Each of R^{L} and x formula (III-I1) correspond to R^{L} and x of formula (III-I). Similar to formula (III-I), hydrogen atoms are implied in the conversion product represented by formula (III-I1), e.g., when x is less than 5.

Illustrative, but non-limiting, examples of suitable substrates for the hydrodefluorination process may include fluorine-containing substrates III-59a and III-60a. Corresponding products from the hydrodefluorination process may include conversion products III-59b and III-60b (Me is methyl).

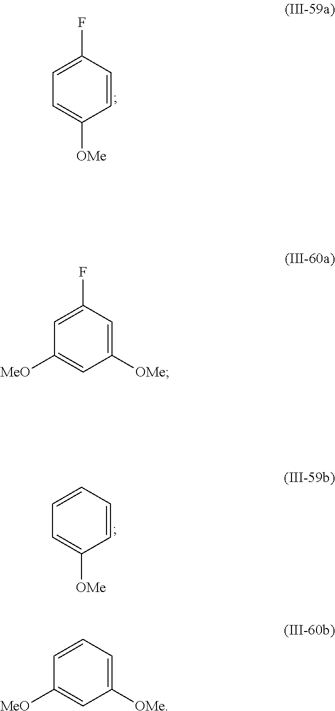 [SMILES: COc1ccc(F)cc1 ; COc1cc(F)cc(OC)c1 ; COc1ccccc1 ; COc1cccc(OC)c1]

The following examples are put forth so as to provide those of ordinary skill in the art with a complete disclosure and description of how to make and use aspects of the present disclosure, and are not intended to limit the scope of aspects of the present disclosure. Efforts have been made to ensure accuracy with respect to numbers used but some experimental errors and deviations should be accounted for.

### Examples

THF, DMF, and DCM were dried using an MBRAUN Inert Gas-System GmbH solvent purification system. Anhydrous DMAc, CH₃CN, methanol (MeOH), and t-AmylOH, were purchased from Sigma Aldrich. All solvents were stored in an N₂-filled glovebox. TMAOH, TBAF, TBACl, and TBABr reductants were purchased from Sigma Aldrich and stored in an N₂-filled glovebox upon receiving. Unless otherwise noted, all substrates and reagents were used as received from commercial suppliers. All experiments were performed under an N₂ atmosphere by using standard Schlenk technique or in a glovebox, if not stated otherwise. All liquid substrates were sparged with N₂ and used in an N₂-filled glovebox.

Photoreactor design. The photoreactor was custom designed. The photoreactor included one light or two lights. Light source specifications for 4 different light sources were investigated: 385 nm (two light); 405 nm (one light); 405 nm (two light); 455 nm (two Kessil lamps). All light-emitting diodes (LEDs) used were purchased from LED Engin. In the photoreactor were used LEDs having a peak wavelength of 405 nm (luminous flux of 4.1 W at 700 mA, Model No. LZ4-40UB0000U8) and LEDs having a peak wavelength of 385 nm (luminous flux of 1.1 W at 700 mA, Model No. LZ1-10UBN0-00U4).

Photoreactor specifications. Besides the light sources, the photoreactor included: a Delta Electronics 60 mm fan (51.7 cfm airflow at 750 mA, 12 V, Part No. QFR-0612DH-B); a Delta Electronics 40 mm fan (20.56 cfm airflow at 430 mA, 12 V, Part #THA0412AD); a Mean Well fan power supply (12 V, 240 W, switching, Part #HLG-240H-12); a Cincon LED power supply (18 V, 150 W, switching, Part #TRH150A180); a LEDdynamics LED driver (Output: Input V—2.5 V, 700 mA, Part #3023-D-N-700); a Wakefield heatsink (2.75″ diameter, 3″ thick aluminum, Part #882-300AB); a 3D-printed reactor chamber (fits 40 mm fan, 65 mm heatsink, made in-house); and a 3D-printed vial holder (0.5 dram, 1.5 dram, or 20 mL, made in-house). 3D printing was accomplished in-house using Autodesk Inventor software and 3D-printed using stereolithography with a Form2 printer (FormLabs) or fused filament fabrication with a Creator Pro (Flashforge). The LEDs were mounted to a metal core printed circuit board (MCPCB) which in turn was mounted to an aluminum heatsink. The MCPCB facilitated efficient heat transfer from the LED to the heatsink. This heatsink was then actively cooled by a 60 mm computer fan. In addition, the reaction chamber was separately cooled by a 40 mm computer fan to facilitate consistent reaction conditions. This chamber also included a reflective interior surface coating comprising aluminum tape to maximize reflection of emitted light back to the reaction vial. A removable vial holder was used to facilitate consistent vial placement at about 5.0 or about 10.0 mm from the LED and allowed for irradiation of 0.5 dram, 1.5 dram, and 20 mL vials (distance from the LED emitter surface to the vial was about 5.0 mm (small-scale reaction) or about 10.0 mm (large-scale reaction)). The LED/MCPCB mounted to the heatsink/fan assembly formed a modular block which allows for facile exchange of LEDs with differing emission wavelengths. An example photoreactor setup may be found in J. Am. Chem. Soc. 2018, 140, 24, 7667-7673.

Analytical thin layer chromatography (TLC) was utilized to monitor reaction progress. Pre-coated Merck glass backed silica gel plates (Silica gel 60 F254) were used for TLC. Visualization was achieved by, e.g., using ultraviolet light (254 nm) or staining with iodine (absorbed on silica gel). Flash column chromatography was performed on Fluka or Material Harvest silica gel (230-400 mesh) under positive air pressure. Flash Combiflash® Rf 150 chromatography purification system was also used to purify the products. Nuclear magnetic resonance (NMR) spectra were recorded at ambient temperature on either a Bruker Ultrashield-400 (400 MHz) spectrometer, a Varian 400 NMR (400 MHz) spectrometer or an Agilent Inova 400 (400 MHz) spectrometer in the Colorado State University Analytical Resources Core (ARC). ¹H-NMR yields and conversions are based on a calibrated area of mesitylene as internal standard. High Resolution Mass spectroscopy (HRMS) analysis was performed on a 6224 Time-of-Flight LC/MS (Agilent) at the Colorado State University Central Instrument Facility.

### Example Catalyst Synthesis

Cat-1, Cat-3, and Cat-4 were made by the following procedure. A 100 mL Schlenk tube was charged with diethyl benzo[ghi]perylene-1,2-dicarboxylate (80 mg, 0.19 mmol, 1.00 eq), 2-(trimethylsilyl)phenyl trifluoromethanesulfonate (177 mg, 0.594 mmol, 3.10 eq) or 4-methoxy-2-(trimethylsilyl)phenyl trifluoromethanesulfonate (195 mg, 0.594 mmol, 3.10 eq) or 4,5-dimethoxy-2-(trimethylsilyl)phenyl trifluoromethanesulfonate (212 mg, 0.594 mmol, 3.10 eq) and cesium fluoride (270 mg, 1.78 mmol, 9.00 eq). To it, THF/CH₃CN (1:1, 9.6 mL) was added and the reaction mixture was stirred at 60° C. for 16 hours. After completion of the reaction, the reaction mixture was cooled to room temperature and concentrated in vacuo. The resulting mixture was extracted with DCM. The crude product was purified by silica-gel column chromatography using hexane:DCM (10:1) to afford the respective compound (Cat-1, Cat-3, or Cat-4) as a yellow solid.

Cat-1 (Diethyl 10,11-dimethoxybenzo[a]coronene-3,4-dicarboxylate): ¹H NMR (400 MHz, CDCl₃) δ 8.31 (dd, J=8.6, 1.5 Hz, 2H), 7.71 (d, J=8.7 Hz, 2H), 7.52 (d, J=8.5 Hz, 2H), 7.29 (d, J=8.4 Hz, 2H), 7.08 (s, 2H), 4.84-4.74 (m, 4H), 4.04 (d, J=1.4 Hz, 6H), 1.70 (td, J=7.2, 1.4 Hz, 6H); ¹³C NMR (101 MHz, CDCl₃) δ 169.1, 148.4, 127.5, 127.2, 126.5, 124.2, 123.5, 123.4, 122.3, 122.0, 121.9, 120.3, 119.6, 118.2, 102.7, 62.1, 55.8, 14.6 ppm. High-resolution mass spectrometry (HRMS) (electrospray ionization (ESI)) Calc. for C₃₆H₂₇O₆ 555.1808 (calculated (calc.)) [M+H]⁺, found 555.1795 (experimental (exptl.)).

Cat-3 (Diethyl benzo[a]coronene-3,4-dicarboxylate): ¹H NMR (400 MHz, CDCl₃) δ 9.26 (d, J=8.8 Hz, 2H), 9.13 (dd, J=6.4, 3.4 Hz, 2H), 8.85 (d, J=8.8 Hz, 2H), 8.56 (dd, J=8.8, 6.0 Hz, 4H), 8.00 (dd, J=6.3, 3.2 Hz, 2H), 4.78 (q, J=7.2 Hz, 4H), 1.66 (t, J=7.2 Hz, 6H); ¹³C NMR (101 MHz, CDCl₃) δ 169.0, 128.6, 127.7, 127.1, 126.3, 123.9, 123, 121.7, 62.3, 14.6 ppm. FIRMS (ESI) Calc. for C₃₄H₂₂O₄ 494.1518 (calc.) [M]⁺, found 494.1525 (exptl.).

Cat-4 (Diethyl 10-methoxybenzo[a]coronene-3,4-dicarboxylate): ¹H NMR (400 MHz, CDCl₃) δ 8.43 (dd, J=11.1, 8.7 Hz, 2H), 8.22 (d, J=9.0 Hz, 1H), 8.15 (d, J=8.6 Hz, 1H), 8.07 (d, J=8.6 Hz, 1H), 7.95 (dd, J=8.8, 2.1 Hz, 2H), 7.70 (dd, J=17.9, 8.5 Hz, 2H), 7.58 (d, J=2.6 Hz, 1H), 7.22 (dd, J=8.9, 2.5 Hz, 1H), 4.80 (q, J=7.2 Hz, 4H), 4.10 (s, 3H), 1.71 (dd, J=7.6, 6.8 Hz, 6H); ¹³C NMR (101 MHz, CDCl₃) δ 169.0, 158.0, 129.0, 128.3, 127.7, 127.5, 127.0, 126.8, 125.2, 124.9, 124.8, 124.6, 124.2, 123.8, 123.6, 123.1, 122.6, 122.4, 122.3, 122.0, 120.7, 120.7, 120.4, 120.2, 120.1, 118.5, 116.4, 103.8, 62.2, 55.5, 14.6 ppm. HRMS: (ESI) Calc. for C₃₅H₂₅O₅ 525.1702 (calc.) [M+H]⁺, found 525.1700 (exptl.).

Cat-2 (Diethyl 7,14-diphenylbenzo[a]coronene-3,4-dicarboxylate) was made by the following procedure. A 2.00 mL scintillation vial was charged with a Teflon-coated stir bar and diethyl 5,7,10-triphenylbenzo[ghi]perylene-1,2-dicarboxylate (9.00 mg, 0.01 mmol, 1.00 eq). Subsequently, the vial was transferred to an N₂-filled glovebox where t-AmylOH (0.40 mL), THF (8.00 mL), and TBAF (11 μL, 0.04 mmol, 4.00 eq (1 M solution in THF)) were added sequentially. The sealed vial was removed from the glovebox and placed into the photoreactor. The reaction mixture was stirred for 9 h under constant 405 nm irradiation inside the photoreactor at room temperature. The reaction was stopped and concentrated in vacuo. The crude mixture was purified by column chromatography using 10:1 hexane:DCM as eluent. ¹H NMR (400 MHz, CDCl₃) δ 9.52 (s, 2H), 9.21 (dd, J=6.4, 3.4 Hz, 2H), 8.96 (d, J=9.2 Hz, 2H), 8.87 (d, J=9.2 Hz, 2H), 7.91 (dd, J=6.4, 3.2 Hz, 2H), 7.89-7.85 (m, 4H), 7.74-7.68 (m, 4H), 7.67-7.61 (m, 2H), 4.71 (q, J=7.2 Hz, 4H), 1.59 (d, J=7.2 Hz, 6H); ¹³C NMR (75 MHz, CDCl₃) δ 168.9, 141.3, 139.1, 131.0, 129.1, 128.8, 128.7, 128.1, 127.9, 127.4, 126.3, 126.2, 124.9, 124.7, 124.3, 123.9, 123.6, 123.4, 121.7, 62.4, 29.8, 14.5 ppm. HRMS (ESI) Calc. for C₄₆H₃₁O₄ 647.2222 (calc.) [M+H]⁺, found 647.2230 (exptl.).

### Example Conversion Processes

Various solvents were tested and included THF, t-AmylOH, DCM, DMF, DMac, CH₃CN, t-AmylOH:THF, H₂O:THF. Various light source wavelengths and photoreactor setups (one light or two lights) were tested and included: 385 nm (two light); 405 nm (one light); 405 nm (two light); 455 nm (two Kessil lamps). Catalyst loading was also investigated at 0.5, 1, 2, 3, and 5 mol % based on the substrate. Various reductants (and amounts of reductants) were tested and included NMe₃, DIPEA, TMAOH, TBAF, TBACl, and TBABr, with amounts tested at, e.g., 5, 11, and 25 equivalents (eq) based on 1 eq of substrate. Other reductants and amounts may be utilized for conversion processes such as those described herein. Other solvents, light source wavelengths, numbers of lights, catalyst loadings, reductants, amounts of reductants, may be utilized such as those described herein.

General procedure for photoredox organocatalyzed aryl reduction. A 1.5-dram scintillation vial was charged with a Teflon-coated stir bar and Cat-1 (2.5 mg, 0.005 mmol, 2.0 mol %). If the substrate is a solid, it was weighed into the 1.5-dram scintillation vial outside the glove box. Subsequently, the vial was transferred to an N₂-filled glovebox where liquid substrate (0.25 mmol, 1.00 eq) was sparged with N₂, and then t-AmylOH (0.50 mL), THF (0.50 mL), and TMAOH (1.0 mL, 2.75 mmol, 11 eq (25% solution in MeOH)) were added sequentially. The sealed vial was removed from the glovebox and placed into the photoreactor. The reaction mixture was stirred for 4 hours under constant irradiation inside the photoreactor with fans placed in line of the vials for cooling (temperatures measured via IR ranged from 25 to 30° C.). The reaction mixture was stopped and concentrated in vacuo. The crude mixture was purified by column chromatography using 10:1 hexane/ethyl acetate as eluent. The desired products were characterized by ¹H NMR, ¹³C NMR, and HRMS.

General procedure for reductions of carbocycles, heterocycles, alkenes/alkynes. A 1.5-dram scintillation vial was charged with a Teflon-coated stir bar and Cat-1 (1.3 mg, 0.0025 mmol, 1 mol %). If the substrate is a solid, it was weighed into the 1.5-dram scintillation vial outside the glove box. Subsequently, the vial was transferred to an N₂-filled glovebox where liquid substrate (0.25 mmol, 1.00 eq) was sparged with N2, and then t-AmylOH (0.50 mL), THF (0.50 mL), and TMAOH (1.0 mL, 2.75 mmol, 11 eq (25% solution in MeOH)) were added sequentially. The sealed vial was removed from the glovebox and placed into the photoreactor. The reaction mixture was stirred for 3 hours under constant irradiation inside the photoreactor with fans placed in line of the vials for cooling (temperatures measured via IR ranged from 25 to 30° C.). The reaction mixture was stopped and concentrated in vacuo. The crude mixture was purified by column chromatography using 10:1 hexane/ethyl acetate as eluent. The desired products were characterized by ¹H NMR, ¹³C NMR, and HRMS.

Conversion of substrates III-1a through III-48a to conversion products III-1b through III-48b. The general procedure was followed, with Cat-1 (1-2 mol %), substrate (0.25 mmol), TMAOH (11 eq (25% solution in MeOH)), 405 nm LEDs (2 lights), solvent of t-AmylOH:THF, and conversion period of 3-4 h. Conversion products (with approximate yield shown in parentheses): III-1b (72%); III-2b (61%); III-3b (52%); III-4b (55%); III-5b (83%); III-6b (59%); III-7b (64%); III-8b (46%); III-9b (40%); III-10b (36%); III-1 1b (80%); III-12b (66%); III-13b (67%); III-14b (81%); III-15b (76%); III-16b (84%); III-17b (75%); III-18b (66%); III-19b (46%); III-20b (71%); III-21b (75%); III-22b (71%); III-23b (71%); III-24b (92%); III-25b (85%); III-26b (92%); III-27b (86%); III-28b (86%); III-29b (93%); III-30b (76%) III-31b (84%); III-32b (76%); III-33b (66%); III-34b (86%); III-35b (79%); III-36b (90%); III-37b (75%); III-38b (65%); III-39b (93%); III-40b (47%); III-41b (89%); III-42b (75%); III-43b (71%); III-44b (84%); III-45b (76%); III-46b (88%); III-47b (72%); and III-48b (63%).

Conversion of substrates III-49a and III-50a to conversion products III-49b and III-50b. The general procedure was followed, with Cat-1 (2 mol %), substrate (0.25 mmol, 1 eq), TMAOH (11 eq (25% solution in MeOH)), 405 nm LEDs (2 lights), solvent of H₂O:THF or t-AmylOH:THF, and a conversion period of 4 h. Conversion products (with approximate yield shown in parentheses): III-49b (30% with 0.1:0.9 H₂O:THF); III-50b (46% with solvent of 1:1 t-AmylOH:THF, or 30% with solvent of 0.1:0.9 H₂O:THF).

Conversion of substrates III-51a and III-52a to conversion products III-51b and III-52b. The general procedure was followed, with Cat-1 (2 mol %), substrate (0.25 mmol, 1 eq), TMAOH (11 eq (25% solution in MeOH)), 405 nm LEDs (2 lights), solvent of t-AmylOH:THF, and a conversion period of 4 h. Conversion products (with approximate yield shown in parentheses): III-51b (77%); III-52b (66%).

Conversion of substrate III-53a, -54b, -55a, and -56a to conversion products III-53b/c, -54b/c, -55b/c, and -56b/c. For selective deoxygenation of III-53a, selective ring-opening of III-54a, hydrodefluorination of III-55a, and selective reduction of III-56a, the general procedure was followed, with Cat-1 (1 mol %), substrate (0.25 mmol, 1 eq), TMAOH (11 eq (25% solution in MeOH)), 405 nm LEDs (2 lights), solvent of t−1:1 AmylOH:THF, and a conversion period of 3 hours. Conversion products (with approximate yield shown in parentheses): III-53b (72%); III-54b (69%); III-55b (85%); III-56b (59%). III-53b, -54b, -55b, or -56b were then subjected to aryl/heteroaryl reduction by following the general procedure, with Cat-1 (2 mol %), substrate (0.25 mmol), TMAOH (11 eq (25% solution in MeOH)), 405 nm LEDs (2 lights), solvent of 1:1 t-AmylOH:THF, and a conversion period of 4 h. Conversion products (with approximate yield shown in parentheses): III-53c (71%); III-54c (75%); III-55c (76%); III-56c (81%).

Alternatively, conversion of substrate III-53a, -54a, -55a, and -56a directly to conversion products III-53c, -54c, -55c, and -56c, respectively, was performed by following the general procedure, with Cat-1 (2 mol %), substrate (0.25 mmol, 1 eq), TMAOH (11 eq (25% solution in MeOH)), 405 nm LEDs (2 lights), solvent of 1:1 t-AmylOH:THF, and a conversion period of 9 h. Conversion product yield: Conversion products (with approximate yield shown in parentheses): III-53c (52%); III-54c (64%); III-55c (59%); III-56c (50%).

Conversion of substrates III-57a (reductive tosylation), III-58a (reductive tosylation), III-59a (hydrodefluorination), III-60a (hydrodefluorination), III-63a (cyclization), III-71a (degradation of amine), III-200a (debenzylation), III-201a (dephosphonation), III-202a (desulfonation), and III-203a (desulfonation). The general procedure was followed, with Cat-1 (1 mol %), substrate (0.25 mmol, 1 eq), TMAOH (2.75 mmol, 11 eq (25% solution in MeOH)), 405 nm LEDs (2 lights), solvent of 1:1 t-AmylOH:THF (0.5 mL:0.5 mL), and a conversion period of 3 h. Conversion products (with approximate yield shown in parentheses): III-57b (80%); III-58b (81%); III-59b (90%); III-60b (25%); III-63b (86%); III-71b (90%); III-200b (70%); III-201b (95%); III-202b (73%); III-203b (99%).

Cross-coupling with substrates III-61a and III-62a. The general procedure was followed, with Cat-1 (1 mol %), substrate (0.25 mmol, 1 eq), TMAOH (2.75 mmol, 11 eq (25% solution in MeOH)), B₂Pin₂ (2.5 eq), 405 nm LEDs (2 lights), solvent of 1:1 t-AmylOH:THF (0.5 mL:0.5 mL), and a conversion period of 3 hours. Conversion products where -A″ is -BPin (pinacolborane) (with approximate yield shown in parentheses): III-61b (53%); III-62b (61%).

Reductive alkylation of substrate III-64a. A 1.5-dram scintillation vial was charged with a Teflon-coated stir bar, Cat-1 (2.5 mg, 0.005 mmol, 2 mol %), and the benzoic acid substrate (30.0 mg, 0.25 mmol, 1.00 eq). Subsequently, the vial was transferred to an N₂-filled glovebox where t-AmylOH (0.50 mL), THF (0.50 mL), and TMAOH (2.75 mmol, 11 eq (25% solution in MeOH)) were added sequentially. The sealed vial was removed from the glovebox and placed into the photoreactor. Methyl Iodide was added dropwise into the reaction mixture via syringe inserted into rubber septum cap over the interval of 2 hours. The reaction mixture was stirred for 4 hours under constant irradiation inside the photoreactor with fans placed in line of the vials for cooling (temperatures measured via IR ranged from 25 to 30° C.). The reaction mixture was stopped and 0.25 mmol of mesitylene (internal standard) was added. Next, 100 μL of the crude mixture was added to 400 μL of CD₃OD and was subjected to ¹H NMR spectroscopy to obtain a ¹H NMR yield for III-64b of 49%.

Photochemical ester degradation of substrate III-65a to conversion products III-65b and III-65c. A 1.5-dram scintillation vial was charged with a Teflon-coated stir bar, Cat-1 (0.0025 mmol, 1 mol %), and 2-phenoxy-1-phenylethan-1-one substrate (38 mg, 0.25 mmol, 1.00 eq). Subsequently, the vial was transferred to an N₂-filled glovebox where t-AmylOH (0.50 mL), THF (0.50 mL), and TMAOH (2.75 mmol, 11 eq (25% solution in MeOH)) were added sequentially. The sealed vial was removed from the glovebox and placed into the photoreactor. The reaction mixture was stirred for 3 hours under constant 405 nm irradiation inside the photoreactor with fans placed in line of the vials for cooling (temperatures measured via IR ranged from 25 to 30° C.). The reaction mixture was stopped, concentrated in vacuo, and purified by column chromatography using 10:1 hexane/ethyl acetate as eluent. Conversion products (with approximate yield shown in parentheses): III-65b (92%); III-65c (89%).

### Aspects of the Disclosure

The present disclosure provides, among others, the following aspects, each of which can be considered as optionally including any alternate aspects:

Aspect 1. A catalyst composition, comprising:
a metal free organic photoredox catalyst comprising a coronenyl compound represented by formula (I):

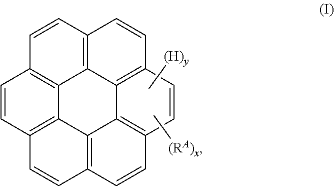

wherein, in formula (I): H is a hydrogen atom; each R^{A} of formula (I) is, independently, a C1-C40 unsubstituted hydrocarbyl, a C1-C40 substituted hydrocarbyl, a functional group comprising at least one element from Group 13-17 of the periodic of the elements, or two or more R^{A} join together to join together to form a monocyclic ring structure or a polycyclic ring structure; x is from 1 to 11, and when x is more than 1, each R^{A} is the same or different; y is from 1 to 11; and x+y is 12.

Aspect 2. The catalyst composition of Aspect 1, wherein the organic photoredox catalyst comprises a benzo[a]coronenyl compound represented by formula (I-A):

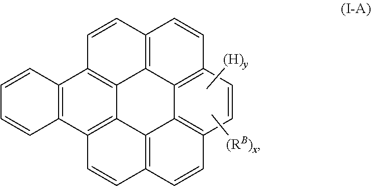

wherein, in formula (I-A): H is a hydrogen atom; each R^{B} of formula (I-A) is, independently, a C1-C20 unsubstituted hydrocarbyl, a C1-C20 substituted hydrocarbyl, a functional group comprising at least one element from Group 13-17 of the periodic of the elements, or two or more R^{B} join together to join together to form a monocyclic ring structure or a polycyclic ring structure; x is from 1 to 13, and when x is more than 1, each R^{B} is the same or different; y is from 1 to 13; and x+y is 14.

Aspect 3. The catalyst composition of any one of the preceding Aspects, wherein the organic photoredox catalyst comprises a benzo[a]coronenyl compound represented by formula (I-A-1):

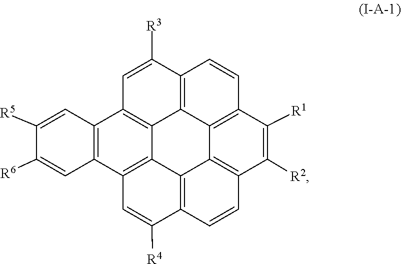

wherein, in formula (I-A-1): each of R¹ and R² of formula (I-A-1) are, independently, a C1-C20 substituted hydrocarbyl (e.g., ester (CO₂Me, CO₂Et), ketone, amide, amine, nitrile), a functional group comprising at least one element from Group 13-17 of the periodic of the elements, or R¹ and R² join together to form a monocyclic ring structure or a polycyclic ring structure; each of R³ and R⁴ are, independently, hydrogen or a C1-C20 unsubstituted hydrocarbyl (e.g., alkyl, aryl, such as Ph); and each of R⁵ and R⁶ are, independently, hydrogen, a C1-C20 unsubstituted hydrocarbyl, or a C1-C20 substituted hydrocarbyl (e.g., C1-C10 alkoxyl (such as OMe)).

Aspect 4. The catalyst composition of any one of the preceding Aspects, wherein the organic photoredox catalyst comprises one or more of:

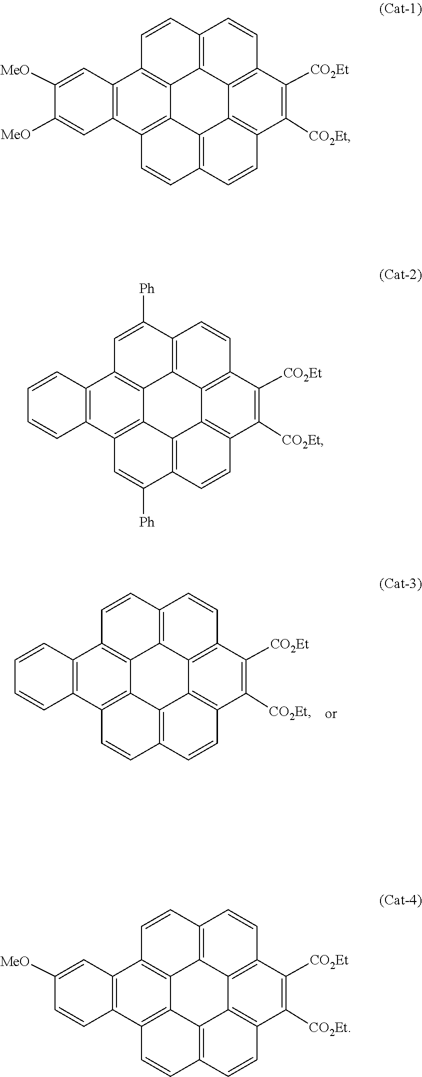 [SMILES: CCOC(=O)c1c(C(=O)OCC)c2ccc3ccc4c5cc(OC)c(OC)cc5c5ccc6ccc1c1c6c5c4c3c21 ; CCOC(=O)c1c(C(=O)OCC)c2ccc3c(-c4ccccc4)cc4c5ccccc5c5cc(-c6ccccc6)c6ccc1c1c2c3c4c5c61 ; CCOC(=O)c1c(C(=O)OCC)c2ccc3ccc4c5ccccc5c5ccc6ccc1c1c6c5c4c3c21 ; CCOC(=O)c1c(C(=O)OCC)c2ccc3ccc4c5cc(OC)ccc5c5ccc6ccc1c1c6c5c4c3c21]

Aspect 5. The catalyst composition of any one of the preceding Aspects, wherein the organic photoredox catalyst is characterized as a catalyst that accepts electrons, donates electrons, or combinations thereof (e.g., a two-electron, 1-proton transfer catalyst).

Aspect 6. The catalyst composition of any one of the preceding Aspects, wherein the catalyst composition further comprises a reductant, such as an amine (e.g., trialkyl or triaryl amines), quaternary ammonium, hydroxide, carboxylic acid, or salt thereof, or combinations thereof, such as TMAOH, NMe₃, DIPEA, TBAF, TBACl, TBABr, a formate salt, or delivered from an electrode or electrochemistry, or combinations thereof.

Aspect 7. The catalyst composition of any one of the preceding Aspects, wherein the catalyst composition further comprises a solvent comprising THF, t-AmylOH, DMF, DCM, toluene, DMAc, CH₃CN, DMSO, water, or combinations thereof.

Aspect 8. The catalyst composition of any one of the preceding Aspects, wherein the catalyst composition catalyzes a reduction of a substrate, wherein: the substrate comprises an aryl group or a heteroaryl group (e.g., indole, benzofuran, benzothiophene, etc.); and the substrate optionally further comprises an alkenyl group, an alkynyl group, an imine group, an azo group, or combinations thereof, chemically bonded to the aryl group or heteroaryl group.

Aspect 9. The catalyst composition of any one of the preceding Aspects, wherein the catalyst composition catalyzes one or more of: reductive detosylation of a substrate, e.g., an arylamine; debenzylation of a substrate, e.g., an aryl ether; dephosphonation of a substrate, e.g., an aryl phosphonate; degradation of a substrate, e.g., a quaternary ammonium salt functional group chemically bonded to an aryl; hydrodefluorination of a substrate, e.g., an arylfluoride; cross-coupling of a substrate, e.g., an organofluoride with a coupling partner to form a coupling product; a cyclization of a substrate, e.g., a diaryl dialdehyde or a diaryl diketone; cleavage of a substrate, e.g., an aryl sulfone; reductive alkylation a substrate, e.g., a benzoic acid; ester fragmentation; lignin fragmentation; or combinations thereof.

Aspect 10. The catalyst composition of any one of the preceding Aspects, wherein the catalyst composition catalyzes a deoxygenation reaction of a substrate such as a ketone.

Aspect 11. The catalyst composition of any one of the preceding Aspects, wherein the catalyst composition catalyzes ring-opening of a substrate, e.g., a cyclopropyl ring.

Aspect 12. The catalyst composition of any one of the preceding Aspects, wherein the catalyst composition catalyzes an at least partial reduction of a substrate, such as an at least partial reduction of an aryl or a heteroaryl of a substrate, e.g., any one of substrates III-101a, III-102a, III-103a, III-104a, III-105a, III-106a, III-107a, III-108a, and/or III-109a.

Aspect 13. The catalyst composition of any one of the preceding Aspects, wherein the catalyst composition catalyzes the reduction of bonds in a substrate, catalyzes the breaking of bonds in a substrate, catalyzes reactions involving a substrate, etc., or combinations thereof in the presence of light having a wavelength (or peak wavelength) in the visible range (e.g., about 350 to about 750 nm), or in a range from about 300 to about 800 nm.

Aspect 13. The catalyst composition of any one of the preceding Aspects, wherein the catalyst composition catalyzes the reduction of bonds in a substrate, catalyzes the breaking of bonds in a substrate, catalyzes reactions involving a substrate, etc., or combinations thereof in the presence of water.

Aspect 14. A conversion process, comprising:
forming a reaction mixture comprising a substrate and a catalyst composition, the catalyst composition comprising:
   an organic photoredox catalyst represented by formula (I-A) (e.g., the organic photoredox catalyst of formula (I-A-1), such as Cat-1, Cat-2, Cat-3, Cat-4, or combinations thereof); and
   a reductant; and
exposing the reaction mixture to light to convert the substrate, under conversion conditions, to a conversion product.

Aspect 15. The conversion process of Aspect 14, wherein the conversion conditions comprise: a conversion temperature in a range from about −10° C. to about 100° C.; a conversion pressure of about 500 kPa or less, or in a range from about 80 to about 120 kPa; a conversion period of about 24 hours or less; or combinations thereof.

Aspect 16. The conversion process of any one of Aspects 14-15, wherein a molar ratio of the organic photoredox catalyst to the substrate is in a range from about 1:10 to about 1:1,000 (organic photoredox catalyst:substrate).

Aspect 17. The conversion process of any one of Aspects 14-16, wherein a molar ratio of the substrate to the reductant is in a range from about 1:0.5 to about 1:50 (substrate:reductant).

Aspect 18. The conversion process of any one of Aspects 14-17, wherein the light a wavelength (or peak wavelength) in the visible range (e.g., about 350 nm to about 750 nm), or in a range from about 300 to about 800 nm.

Aspect 19. The catalyst composition of any one of Aspects 14-18, further comprising a reductant, such as an amine (e.g., trialkyl or triaryl amines), quaternary ammonium, hydroxide, carboxylic acid, or salt thereof, or combinations thereof, such as TMAOH, NMe₃, DIPEA, TBAF, TBACl, TBABr, a formate salt, or delivered from an electrode or electrochemistry, or combinations thereof.

Aspect 20. The conversion process of any one of Aspects 14-19, wherein the reaction mixture further comprises a solvent.

Aspect 21. The conversion process of Aspect 20, wherein the solvent comprises THF, t-AmylOH, DMF, DCM, toluene, DMAc, CH₃CN, DMSO, water, or combinations thereof.

Aspect 22. The conversion process of any one of Aspects 14-21, wherein the substrate comprises a C6-C40 aryl (e.g., phenyl, naphthyl, fluorenyl) or a C5-C40 heteroaryl (e.g., indole, benzofuran, benzothiophene).

Aspect 23. The conversion process of Aspect 22, wherein the C6-C40 aryl or the C5-C40 heteroaryl is substituted with hydrogen, an unsubstituted hydrocarbyl, a substituted hydrocarbyl, or a functional group comprising at least one element from Group 13-17 of the periodic table of the elements, or combinations thereof.

Aspect 24. The conversion process of any one of Aspects 14-23, wherein the substrate is represented by formula (III-A):

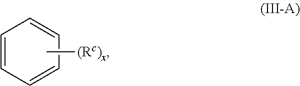

wherein, in formula (III-A): each R^{C} is, independently, a C1-C40 unsubstituted hydrocarbyl, a C1-C40 substituted hydrocarbyl, a functional group comprising at least one element from Group 13-17 of the periodic table of the elements, or two or more R^{C} join together to join together to form a monocyclic ring structure or a polycyclic ring structure; and x is 0 or more, and when x is more than 1, each R^{C} is the same or different.

Aspect 25. The conversion process of Aspect 24, wherein:
each R^{C} of formula (III-A) is, independently:
   a C1-C20 unsubstituted hydrocarbyl, such as a C1-C10 unsubstituted hydrocarbyl, such as:
      a C1-C10 alkyl, such as C1-C6 alkyl, e.g., methyl, ethyl, n-propyl, isopropyl, n-butyl, iso-butyl, sec-butyl, tert-butyl;
      a C2-C10 alkenyl, such as C2-C6 alkenyl, e.g., ethenylene, propenylene, 2-butenylene; or
      a C2-C10 alkynyl, e.g., C2-C6 alkynyl, such as propargyl;
a C1-C20 substituted hydrocarbyl, such as a C1-C10 substituted hydrocarbyl, such as a C1-C6 substituted hydrocarbyl, e.g., wherein at least one hydrogen of the substituted hydrocarbyl is substituted with halogen, NO₂, OR*, C(O)R*, C(O)NR*₂, C(O)OR*, NR*₂, SR*, PR*₂, P(O)R*₂, SO₂R*, SO₃R*, wherein each R* is hydrogen or hydrocarbyl (such as unsubstituted hydrocarbyl or substituted hydrocarbyl).

Aspect 26. The conversion process of any one of Aspects 14-25, wherein the substrate comprises one or more of substrates III-1a through III-30a.

Aspect 27. The conversion process of Aspect 26, wherein the conversion product comprises one or more of conversion products III-1b through III-30b.

Aspect 28. The conversion process of any one of Aspects 14-25, wherein the substrate comprises one or more of substrates III-31a through III-48a.

Aspect 29. The conversion process of Aspect 28, wherein the conversion product comprises one or more of conversion products III-31b through III-48b.

Aspect 30. The conversion process of any one of Aspects 14-25, wherein the substrate comprises one or more of substrates III-49a through III-56a.

Aspect 31. The conversion process of Aspect 30, wherein the conversion product comprises one or more of conversion products III-49b, III-50b, III-51b, III-52b, III-53b, III-53c, III-54b, III-54c, III-55b, III-55c, III-56b, and/or III-56c.

Aspect 32. The conversion process of any one of Aspects 14-31, wherein:
the substrate comprises: (a) an aryl group or a heteroaryl group; and (b) an alkynyl group, an alkenyl group, an imine group, or an azo group chemically bonded to the aryl or heteroaryl group; and
the organic photoredox catalyst represented by formula (I-A) (e.g., the organic photoredox catalyst of formula (I-A-1), such as Cat-1) selectively reduces the alkynyl group, alkenyl group, imine group, or azo group.

Aspect 33. The conversion process of Aspect 32, wherein the organic photoredox catalyst represented by formula (I-A) (e.g., the organic photoredox catalyst of formula (I-A-1), such as Cat-1), at least partially reduces the aryl group or heteroaryl group after reduction of the alkynyl group, the alkenyl group, the imine group, or the azo group.

Aspect 34. The conversion process of any one of Aspects 14-25, wherein:
the substrate is represented by formula (III-B):

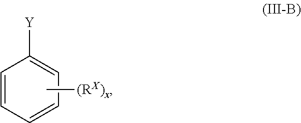

wherein, in formula (III-B): Y comprises an alkoxyl, a phosphonyl, or a sulfonyl; R^{X} is a C1-C20 unsubstituted hydrocarbyl, a C1-C20 substituted hydrocarbyl, a functional group comprising at least one element from Group 13-17 of the periodic of the elements, or two or more R^{X} join together to join together to form a monocyclic ring structure or a polycyclic ring structure; and x is an integer from 0 to 5, and when x is more than 1, each R^{X} is the same or different; and
the conversion product is represented by formula (III-B1):

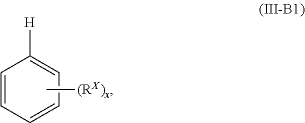

wherein, in formula (III-B): R^{X} and x are the same as R^{X} and x of formula (III-B).

Aspect 35. The conversion process of Aspect 34, wherein the substrate comprises one or more of substrates III-200a, III-201a, III-202a, and/or III-203a.

Aspect 36. The conversion process of any one of Aspects 14-35, wherein the conversion process is used for: reductive detosylation of a substrate such as an arylamine (such as an indole); debenzylation of a substrate such as an aryl ether; dephosphonation of a substrate such as an aryl phosphonate; degradation of a substrate such as a quaternary ammonium salt functional group chemically bonded to an aryl; hydrodefluorination of a substrate such as an arylfluoride; cross-coupling of a substrate such as an organofluoride with a coupling partner to form a coupling product; a cyclization of a substrate such as a diaryl dialdehyde or a diaryl diketone; cleavage of a substrate such as an aryl sulfone; reductive alkylation a substrate such as a benzoic acid; ester fragmentation; lignin fragmentation; or combinations thereof.

Aspect 37. The conversion process of Aspect 36, wherein: the conversion process is used for detosylation of an indole; the substrate comprises substrate(s) III-57a and/or III-58a; and/or the conversion product comprises conversion product(s) III-57b and/or III-58b.

Aspect 38. The conversion process of Aspect 36, wherein: the conversion process is used for degradation of the quaternary ammonium salt functional group chemically bonded to the aryl; the substrate comprises substrate III-71a; and/or the conversion product comprises conversion product III-71b.

Aspect 39. The conversion process of Aspect 36, wherein: the conversion process is used for cyclization of a diaryl dialdehyde; the substrate comprises substrate III-63a; and/or the conversion product comprises conversion product III-63b.

Aspect 40. The conversion process of Aspect 36, wherein: the conversion process is used for cross-coupling of an organofluoride to form a coupling product (such as a boron-containing coupling product); the substrate comprises substrate(s) III-61a and/or III-62a; and/or the conversion product comprises conversion product(s) III-61b and/or III-62b.

Aspect 41. The conversion process of Aspect 36, wherein: the conversion process is used for reductive alkylation of benzoic acid; the substrate comprises benzoic acid; and/or the conversion product comprises conversion product III-64b.

Aspect 42. The conversion process of Aspect 36, wherein: the conversion process is used for ester fragmentation; the substrate comprises substrate III-65a; and/or the conversion product comprises conversion product(s) III-65b and/or III-65c.

Aspect 43. A hydrodefluorination process, comprising:
forming a reaction mixture comprising a fluorine-containing substrate and a catalyst composition, the catalyst composition comprising: (a) an organic photoredox catalyst represented by formula (I-A) (e.g., the organic photoredox catalyst of formula (I-A-1), such as Cat-1, Cat-2, Cat-3, Cat-4, or combinations thereof); and (b) a reductant; and
exposing the reaction mixture to light to convert the substrate, under conversion conditions, to a conversion product.

Aspect 44. The hydrodefluorination process of Aspect 43, wherein the hydrodefluorination process comprises any one of Aspects 14-42.

Aspect 45. The hydrodefluorination process of any one of Aspects 43-44, wherein:
the fluorine-containing substrate is represented by formula (III-I):

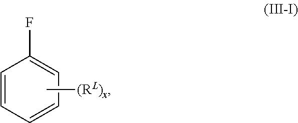

wherein in formula (III-I): each R^{L} is, independently, a C1-C20 unsubstituted hydrocarbyl, a C1-C20 substituted hydrocarbyl, a functional group comprising at least one element from Group 13-17 of the periodic of the elements, or two or more R^{L} join together to join together to form a monocyclic ring structure or a polycyclic ring structure; and x is from 0 to 5, and when x is more than 1, each R^{L} is the same or different; and the conversion product is represented by formula (III-I1):

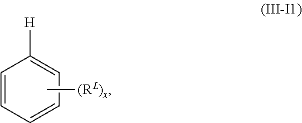

wherein, in formula (III-I1): R^{L} and x are the same as R^{L} and x of formula (III-I).

Aspect 46. The hydrodefluorination process of any one of Aspects 43-45, wherein the fluorine-containing substrate comprises one or more of substrates III-59a and/or III-60a.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), may be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Generally, groups of elements are indicated using the numbering scheme indicated in the version of the periodic table of elements published in Chemical and Engineering News, 63(5), 27, 1985. In some instances, a group of elements may be indicated using a common name assigned to the group; for example, alkali metals for Group 1 elements, alkaline earth metals for Group 2 elements, transition metals for Group 3-12 elements, and halogens or halides for Group 17 elements.

“Hydrocarbon” refers to a compound containing only carbon and hydrogen. The term “hydrocarbyl group” is used herein in accordance with the definition specified by IUPAC: a univalent group formed by removing a hydrogen atom from a hydrocarbon (that is, a group containing only carbon and hydrogen). Other identifiers may be utilized to indicate the presence of particular groups in the hydrocarbon (e.g., halogenated hydrocarbon indicates the presence of one or more halogen atoms replacing an equivalent number of hydrogen atoms in the hydrocarbon). Hydrocarbyl groups may be linear or branched, saturated or unsaturated, cyclic or acyclic, aromatic or non-aromatic. Regarding saturation, hydrocarbyl groups may be fully saturated, partially unsaturated, or fully unsaturated.

A group or groups may be referred to herein as “unsubstituted” or by equivalent terms such as “non-substituted,” which refers to the original group (e.g., a hydrocarbyl, an R group, a ring, or other group) in which a non-hydrogen moiety does not replace a hydrogen within that group. For example, an unsubstituted R group refers to an R group that consists of hydrogen and carbon atoms only. “Unsubstituted hydrocarbyl” includes groups having a C1-C100 group and may be linear or branched, acyclic or cyclic, saturated or unsaturated. When cyclic, the unsubstituted hydrocarbyl may be aromatic or non-aromatic. Regarding saturation, the unsubstituted hydrocarbyl may be fully saturated, partially unsaturated, or fully unsaturated. Examples of unsubstituted hydrocarbyl groups may include alkyl, alkenyl, aryl, and aralkyl groups, amongst other groups. Non-limiting examples of unsubstituted hydrocarbyl may include a group having from 1 to 100 carbon atoms, such as from 1 to 30 carbon atoms, such as from 1 to 20 carbon atoms, such as from 3 to 10 carbon atoms, such as methyl, ethyl, n-propyl, isopropyl, n-butyl, iso-butyl, sec-butyl, tert-butyl, pentyl, hexyl, heptyl, octyl, ethyl-2-hexyl, isooctyl, butyl-2-octyl, nonyl, n-decyl, isodecyl, or isomers thereof, a cycloaliphatic group having from 3 to 40 carbon atoms, such as from 3 to 20 carbon atoms such as, for example, cyclopentyl or cyclohexyl; an alkenyl group having from 2 to 40 carbon atoms, such as from 2 to 20 carbon atoms such as propenyl, butenyl, pentenyl, or hexenyl, an aromatic group having from 6 to 38 carbon atoms, such as from 6 to 20 carbon atoms such as, for example, phenyl, tolyl, or naphthyl; or any combination thereof.

The term “substituted” when used to describe a group (e.g., a hydrocarbyl, an R group, a ring, or other group), for example, when referring to a substituted analog of a particular group, is intended to describe any non-hydrogen moiety that formally replaces a hydrogen in that group, and is intended to be non-limiting. Unless otherwise specified, “substituted” is intended to be non-limiting and include suitable inorganic substituents or organic substituents as understood by one of ordinary skill in the art. For example, a substituted R group or substituted hydrocarbyl refers to a group where at least one hydrogen of the R group or the unsubstituted hydrocarbyl has been substituted with at least one heteroatom or heteroatom-containing group, such as a halogen (F, Cl, Br, or I) or one or more elements from Group 13-16 of the periodic table of the elements, such as O, N, Se, Te, P, As, Sb, S, B, Si, Ge, Sn, Pb, and the like, such as C(O)R*, C(O)NR*₂, C(O)OR*, NR*₂, OR*, SeR*, TeR*, PR*₂, AsR*₂, SbR*₂, SR*, SOx (where x=2 or 3), BR*₂, SiR*₃, GeR*₃, SnR*3, PbR*₃, and the like, where R* is, independently, hydrogen or hydrocarbyl, or where at least one heteroatom has been inserted within the R group or the unsubstituted hydrocarbyl. Substituted hydrocarbyl groups may be linear or branched, saturated or unsaturated, cyclic or acyclic, aromatic or non-aromatic. Regarding saturation, substituted hydrocarbyl groups may be fully saturated, partially unsaturated, or fully unsaturated.

A functional group comprising at least one element from Group 13-17 of the periodic table of the elements may include a halogen (F, Cl, Br, or I), O, N, Se, Te, P, As, Sb, S, B, Si, Ge, Sn, Pb, and the like, such as C(O)R*, C(O)NR*₂, C(O)OR*, NR*₂, OR*, SeR*, TeR*, PR*₂, AsR*₂, SbR*₂, SR*, SOx (where x=2 or 3), BR*₂, SiR*₃, GeR*₃, SnR*₃, PbR*₃, and the like, wherein each R* is, independently, hydrogen or hydrocarbyl. Non-limiting examples of functional groups that replace one or more hydrogen atoms may, independently, include —OH, —F, —Cl, —Br, —I, —NH₂, —NO₂, —N₃, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

“Alkenyl” and “alkenyl group” refer to a straight or branched hydrocarbon chain group containing at least one double bond. The alkenyl may be an unsubstituted alkenyl (carbon and hydrogens only) or a substituted alkenyl (comprising at least one heteroatom or one heteroatom-containing group from Group 13-17 of the periodic table of the elements). “Alkynyl” refers to a straight or branched hydrocarbon chain group containing at least one triple bond. The alkynyl may be an unsubstituted alkynyl (carbon and hydrogens only) or a substituted alkynyl (comprising at least one heteroatom or one heteroatom-containing group from Group 13-17 of the periodic table of the elements).

Reference to chemical compounds without specifying a particular isomer (such as butanol) expressly discloses all isomers (such as n-butanol, iso-butanol, sec-butanol, and tert-butanol). For example, reference to a chemical compound having 4 carbon atoms expressly discloses all isomers thereof. When a compound is described herein such that a particular isomer, enantiomer, or diastereomer of the compound is not specified, e.g., in a formula or in a chemical name, that description is intended to include each isomer, diastereomer, and enantiomer of the compound described individually or in any combination.

As is apparent from the foregoing general description and the specific aspects, while forms of the aspects have been illustrated and described, various modifications may be made without departing from the spirit and scope of the present disclosure. Accordingly, it is not intended that the present disclosure be limited thereby. Likewise, the term “comprising” is considered synonymous with the term “including.” Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “Is” preceding the recitation of the composition, element, or elements and vice versa, such as the terms “comprising,” “consisting essentially of,” “consisting of” also include the product of the combinations of elements listed after the term.

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. For example, the recitation of the numerical range 1 to 5 includes the subranges 1 to 4, 1.5 to 4.5, 1 to 2, among other subranges. As another example, the recitation of the numerical ranges 1 to 5, such as 2 to 4, includes the subranges 1 to 4 and 2 to 5, among other subranges. Additionally, within a range includes every point or individual value between its end points even though not explicitly recited. For example, the recitation of the numerical range 1 to 3 includes the numbers 1, 1.5, 2, 2.75, 3, among other numbers. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, aspects comprising “a solvent” includes aspects comprising one, two, or more solvents, unless specified to the contrary or the context clearly indicates only one solvent is included.

While the foregoing is directed to aspects of the present disclosure, other and further aspects of the disclosure may be devised without departing from the basic scope thereof, and the scope thereof is determined by the claims that follow.

What is claimed is:

1. A catalyst composition, comprising:
a metal free organic photoredox catalyst comprising a coronenyl compound represented by formula (I): 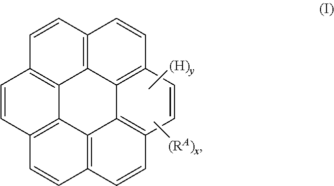
wherein:
each H of formula (I) is a hydrogen atom;
each R^{A} of formula (I) is, independently, a C1-C40 unsubstituted hydrocarbyl, a C1-C40 substituted hydrocarbyl, a functional group comprising at least one element from group 13-17 of the periodic of the elements, or two or more R^{A}join together to join together to form a monocyclic ring structure or a polycyclic ring structure;
x of formula (I) is from 1 to 11, and when x is more than 1, each R^{A} is the same or different;
y of formula (I) is from 1 to 11; and
x+y is 12.

2. The catalyst composition according to claim 1, wherein the organic photoredox catalyst comprising the coronenyl compound represented by formula (I) comprises a benzo[a]coronenyl compound represented by formula (I-A): 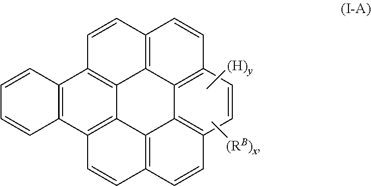
wherein:
each H of formula (I-A) is a hydrogen atom;
each R^{B} of formula (I-A) is, independently, a C1-C20 unsubstituted hydrocarbyl, a C1-C20 substituted hydrocarbyl, a functional group comprising at least one element from group 13-17 of the periodic of the elements, or two or more R^{B} join together to join together to form a monocyclic ring structure or a polycyclic ring structure;
x of formula (I-A) is from 1 to 13, and when x is more than 1, each R^{B} is the same or different;
y of formula (I-A) is from 1 to 13; and
x+y is 14.

3. The catalyst composition according to claim 1, wherein the organic photoredox catalyst comprising the coronenyl compound represented by formula (I) comprises a benzo[a]coronenyl compound represented by formula (I-A-1): 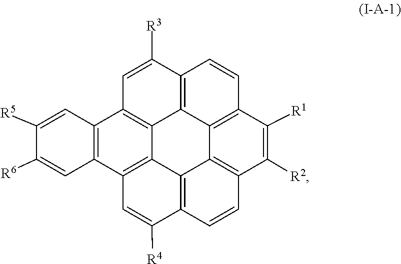
wherein:
each of R¹ and R² of formula (I-A-1) are, independently, a C1-C20 substituted hydrocarbyl, a functional group comprising at least one element from group 13-17 of the periodic of the elements, or R¹ and R² join together to form a monocyclic ring structure or a polycyclic ring structure;
each of R³ and R⁴ of formula (I-A-1) are, independently, hydrogen or a C1-C20 unsubstituted hydrocarbyl; and
each of R⁵ and R⁶ of formula (I-A-1) are, independently, hydrogen, a C1-C20 unsubstituted hydrocarbyl, or a C1-C20 substituted hydrocarbyl.

4. The catalyst composition according to claim 1, wherein the organic photoredox catalyst comprises one or more of:

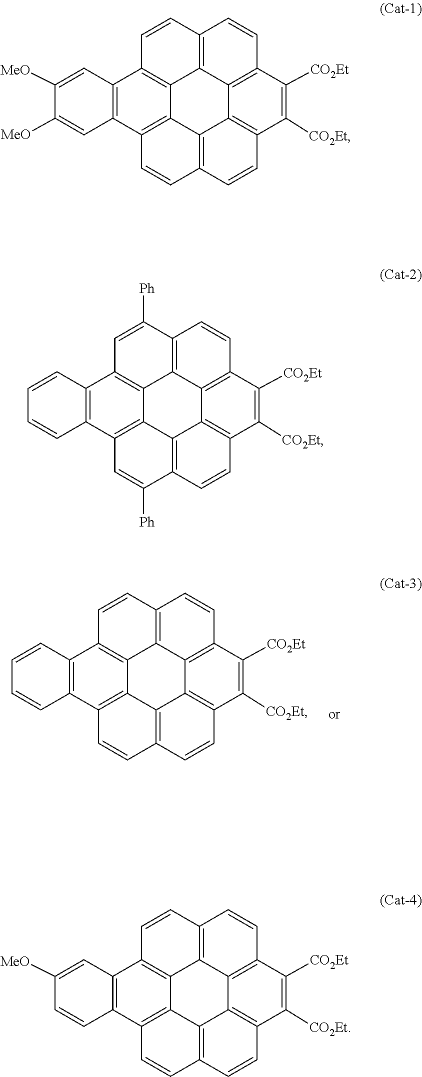 [SMILES: CCOC(=O)c1c(C(=O)OCC)c2ccc3ccc4c5cc(OC)c(OC)cc5c5ccc6ccc1c1c6c5c4c3c21 ; CCOC(=O)c1c(C(=O)OCC)c2ccc3c(-c4ccccc4)cc4c5ccccc5c5cc(-c6ccccc6)c6ccc1c1c2c3c4c5c61 ; CCOC(=O)c1c(C(=O)OCC)c2ccc3ccc4c5ccccc5c5ccc6ccc1c1c6c5c4c3c21 ; CCOC(=O)c1c(C(=O)OCC)c2ccc3ccc4c5cc(OC)ccc5c5ccc6ccc1c1c6c5c4c3c21]

5. The catalyst composition according to claim 1, wherein the catalyst composition further comprises a reductant.

6. The catalyst composition according to claim 5, wherein:
the reductant comprises an amine, a quaternary ammonium, a hydroxide, a carboxylic acid, a salt thereof, or combinations thereof;
the reductant is delivered by an electrode; or
both.

7. The catalyst composition according to claim 5, wherein the reductant comprises tetramethylammonium hydroxide, trimethylamine, N,N-diisopropylethylamine, tetra-n-butylammonium fluoride, tetra-n-butylammonium chloride, tetra-n-butylammonium bromide, a salt of formic acid, or combinations thereof.

8. The catalyst composition according to claim 1, wherein the catalyst composition further comprises a solvent.

9. The catalyst composition according to claim 8, wherein the solvent comprises tetrahydrofuran, tert-amyl alcohol, dimethylformamide, dichloromethane, toluene, dimethylacetamide, acetonitrile, dimethyl sulfoxide, water, or combinations thereof.

10. The catalyst composition according to claim 1, wherein the catalyst composition catalyzes reactions in the presence of light having a wavelength (or peak wavelength) in the visible range.

11. The catalyst composition according to claim 1, wherein the catalyst composition catalyzes reactions in the presence of water.

12. A conversion process, comprising:
forming a reaction mixture comprising a substrate and a catalyst composition, the catalyst composition comprising:
a reductant; and
an organic photoredox catalyst comprising a benzo[a]coronenyl compound represented by formula (I-A): 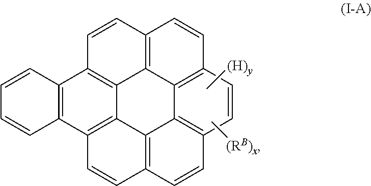
wherein:
each H of formula (I-A) is a hydrogen atom;
each R^{B} of formula (I-A) is, independently, a C1-C20 unsubstituted hydrocarbyl, a C1-C20 substituted hydrocarbyl, a functional group comprising at least one element from group 13-17 of the periodic of the elements, or two or more R^{B} join together to join together to form a monocyclic ring structure or a polycyclic ring structure;
x of formula (I-A) is from 1 to 13, and when x is more than 1, each R^{B} is the same or different;
y of formula (I-A) is from 1 to 13; and
x+y is 14; and
exposing the reaction mixture to light to convert the substrate, under conversion conditions, to a conversion product.

13. The conversion process according to claim 12, wherein the benzo[a]coronenyl compound represented by formula (I-A) comprises the benzo[a]coronenyl compound represented by formula (I-A-1): 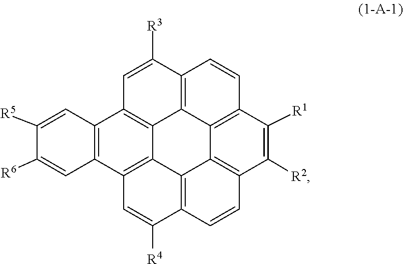
wherein:
each of R¹ and R² of formula (I-A-1) are, independently, a C1-C20 substituted hydrocarbyl, a functional group comprising at least one element from group 13-17 of the periodic of the elements, or R¹ and R² join together to form a monocyclic ring structure or a polycyclic ring structure;
each of R³ and R⁴ of formula (I-A-1) are, independently, hydrogen or a C1-C20 unsubstituted hydrocarbyl; and
each of R⁵ and R⁶ of formula (I-A-1) are, independently, hydrogen, a C1-C20 unsubstituted hydrocarbyl, or a C1-C20 substituted hydrocarbyl.

14. The conversion process according to claim 12, wherein the substrate comprises an aryl, an unsubstituted aryl, a substituted aryl, a heteroaryl, an unsubstituted heteroaryl, a substituted heteroaryl, or combinations thereof.

15. The conversion process according to claim 12, wherein the light has a wavelength in a range from about 300 nm to about 800 nm.

16. The conversion process according to claim 12, wherein the conversion conditions comprise:
a conversion temperature in a range from about −10° C. to about 100° C.;
a conversion pressure of about 500 kPa or less;
a conversion period of about 24 hours or less; or
combinations thereof.

17. The conversion process according to claim 12, wherein the reductant comprises tetramethylammonium hydroxide, trimethylamine, N,N-diisopropylethylamine, tetra-n-butylammonium fluoride, tetra-n-butylammonium chloride, tetra-n-butylammonium bromide, a salt of formic acid, or combinations thereof.

18. A conversion process, comprising:
forming a reaction mixture comprising a fluorine-containing substrate and a catalyst composition, the catalyst composition comprising:
a reductant; and
an organic photoredox catalyst comprising a benzo[a]coronenyl compound represented by formula (I-A): 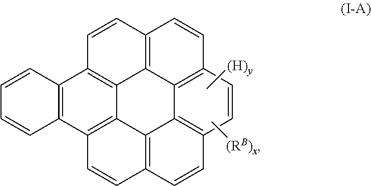
wherein:
each H of formula (I-A) is a hydrogen atom;
each R^{B} of formula (I-A) is, independently, a C1-C20 unsubstituted hydrocarbyl, a C1-C20 substituted hydrocarbyl, a functional group comprising at least one element from group 13-17 of the periodic of the elements, or two or more R^{B} join together to join together to form a monocyclic ring structure or a polycyclic ring structure;
x of formula (I-A) is from 1 to 13, and when x is more than 1, each R^{B} is the same or different;
y of formula (I-A) is from 1 to 13; and
x+y is 14; and
exposing the reaction mixture to light to convert the substrate, under conversion conditions, to a conversion product.

19. The conversion process according to claim 18, wherein:
the fluorine-containing substrate is represented by formula (III-I): 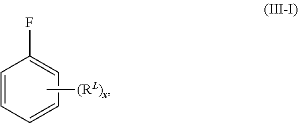
wherein:
R^{L} of formula (III-I) is, independently, a C1-C20 unsubstituted hydrocarbyl, a C1-C20 substituted hydrocarbyl, a functional group comprising at least one element from group 13-17 of the periodic of the elements, or two or more R^{L} join together to join together to form a monocyclic ring structure or a polycyclic ring structure; and
x of formula (III-I) is from 0 to 5, and when x is more than 1, each R^{L} is the same or different; and
the conversion product is represented by formula (III-I1): 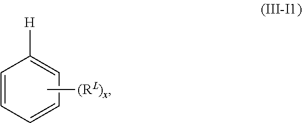
wherein R^{L} and x of formula (III-I1) are the same as R^{L} and x of formula (III-I).

20. The conversion process according to claim 18, wherein:
the fluorine-containing substrate is represented by formula (III-F): 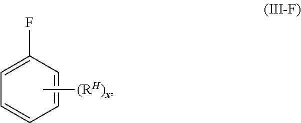
wherein:
each R^{H} of formula (III-F) is, independently, a C1-C20 unsubstituted hydrocarbyl, a C1-C20 substituted hydrocarbyl, a functional group comprising at least one element from Group 13-17 of the periodic table of the elements, or two or more R^{H} join together to join together to form a monocyclic ring structure or a polycyclic ring structure; and
x of formula (III-I) is from 0 to 5, and when x is more than 1, each R^{H} is the same or different; and
the conversion product is represented by formula (III-I1): 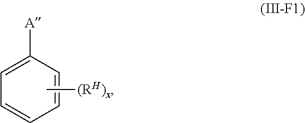
wherein:
each of R^{H} and x of formula (III-F1) are the same as R^{L} and x of formula (III-F1); and
A″ of formula (III-F1) is a boron-containing group.
